(19) United States
(12) Patent Application Publication
(10) Pub. No.: US 2013/0184684 A1
(43) Pub. Date: Jul. 18, 2013

(54) NASAL CLEANING APPRATUSES, KITS, AND METHODS OF USE

(71) Applicant: Mark J. Yardley, Beaver, UT (US)

(72) Inventor: Mark J. Yardley, Beaver, UT (US)

(21) Appl. No.: 13/551,463

(22) Filed: Jul. 17, 2012

Related U.S. Application Data
(63) Continuation-in-part of application No. 12/408,654, filed on Mar. 20, 2009, now Pat. No. 8,241,236.
(60) Provisional application No. 61/038,476, filed on Mar. 21, 2008.

(51) Int. Cl. A61F 13/12; A61M 31/00

(52) U.S. Cl. CPC A61F 13/126; A61M 31/00
USPC 604/514; 606/162

(57) ABSTRACT

A nasal cleaning apparatus is described that may include a nasal cleaning portion and a grip portion. The nasal cleaning portion may include a proximal end portion, a distal end portion, and a front wall extending between the proximal end portion and the distal end portion, and a back wall. The back wall may be attached to the front wall along at least a portion of the periphery of the front wall and/or back wall. An internal space may be at least partially defined between the front wall and the back wall. An opening may be in the proximal end portion. The opening may be in communication with the internal space. The grip portion may include a handle and an extension member. The extension member may extend from a distal end portion of said handle, through the opening of the nasal cleaning portion, and into the internal space of the nasal cleaning portion.

### CROSS-REFERENCE TO RELATED APPLICATIONS

This application is a continuation-in-part of U.S. patent application Ser. No. 12/408,654 entitled “Apparatus for Cleaning a Nasal Cavity,” filed Mar. 20, 2009, which claims priority to and the benefit of U.S. Provisional Application No. 61/038,476, entitled “Nasal Swab,” filed on Mar. 21, 2008. Each of the aforementioned applications is hereby incorporated herein in its entirety by this reference.

### BACKGROUND OF THE INVENTION

1. The Field of the Invention

Exemplary embodiments of the invention relate to devices and methods for cleaning the nose. More particularly, exemplary embodiments of the invention relate to devices and methods for cleaning nasal cavities to remove substances therein.

2. The Relevant Technology

Human beings and other animals generally suffer from all sorts of ailments which can be treated or prevented by taking proper remedial or preventative measures. For example, a person who comes into contact with dust, smoke, smog, pollen, pet danger, debris, germs, bacteria, a virus, and the like may suffer from allergies, the common cold, the flu, a bacterial infection, or other ailments. However, if these substances and organisms can be removed in a timely manner, the person may be able to prevent such ailments from occurring, or at least minimize the effects of such substances.

Removing these substances and organisms is oftentimes not an easy task, particularly inasmuch as dust, bacteria and viruses may be received in a bodily orifice, such as the nostrils of the nose, and then internalized such that regular cleaning of the body of the person will not remove these ailment causing materials. However, before such materials are fully internalized, they may enter the nose and attach to the walls of the nasal cavity and/or hairs or other items therein. When such materials attach to the walls of the nasal cavity, or are internalized after entering the nasal cavity, the materials may be difficult to remove even if the person blows his or her nose. If, however, such attached materials can be quickly and easily removed, a person may be able to prevent such materials from causing an ailment, and/or may be able to remedy an ailment already being suffered. By quickly and efficiently removing such materials, a person may thus avoid or minimize colds, the flu, allergies, and the like. By minimizing such ailments, people may, for example, lose less time at work, and/or save money that would otherwise be spent on over-the-counter or prescription drugs, and generally reduce the strain on their immune systems.

Accordingly, what is desired is an apparatus that safely, efficiently and sanitarily removing debris and other materials from inside of a person's nose.

### BRIEF SUMMARY OF THE INVENTION

Exemplary embodiments of the invention relate to apparatus and assemblies for cleaning nostrils and nasal cavities. More particularly, exemplary embodiments relate to devices and methods for cleaning nasal cavities to remove dust, pollen, germs, and other substances from therein.

In an embodiment a nasal cleaning apparatus may include a nasal cleaning portion configured to be inserted into a nostril to help moisten and/or clean the nostril and a grip portion. The nasal cleaning portion may include a proximal end portion, a distal end portion, and a front wall extending between the proximal end portion and the distal end portion, and a back wall. The back wall may be attached to the front wall along at least a portion of the periphery of the front wall and/or back wall. An internal space may be at least partially defined between the front wall and the back wall. An opening may be in the proximal end portion. The opening may be in communication with the internal space. The grip portion may include a handle and an extension member. The extension member may extend from a distal end portion of said handle, through the opening of the nasal cleaning portion, and into the internal space of the nasal cleaning portion.

In another embodiment, a nasal cleaning kit may include a nasal cleaning apparatus and a storage tube. The nasal cleaning apparatus may include a nasal cleaning portion comprising a sock-like body including a proximal end portion and a distal end portion. The nasal cleaning portion may further include an internal space with said sock-like body. The nasal cleaning portion may also include an opening formed in the proximal end portion that is in communication with the internal space. The nasal cleaning apparatus may also include a grip portion including a handle and an extension member connected to the handle. At least a portion of the extension member may extend through the opening of the nasal cleaning portion into the internal space of the nasal cleaning portion. The storage tube may be removably attached to the handle of the grip portion. The storage tube may include a lumen extending at least partially through the storage tube. The lumen may be configured to selectively receive and store at least the nasal cleaning portion of the nasal cleaning apparatus.

In another embodiment, a method for moistening and/or cleaning a nasal cavity may include providing any one the nasal cleaning apparatuses described above. The method may further include grasping the grip portion of the nasal cleaning apparatus in a user's hand and/or fingers and twisting the nasal cleaning portion of the nasal cleaning apparatus about the extension member. The method may also include inserting at least a portion of the nasal cleaning portion into a nasal cavity and rotating the nasal cleaning portion with the extension member to at least partially moisten and/or clean the nasal cavity. Finally, the method may include removing the nasal cleaning portion from the nasal cavity. In other embodiments, the method may further include moistening the nasal cleaning portion with at least one of water, moisturizer, or saline solution before inserting the nasal cleaning portion into the nasal cavity.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope, nor are the drawings necessarily drawn to scale. The invention will be described and explained with additional specificity and detail through the use of the accompanying drawings in which:

FIG. 1 illustrates a perspective view of an exemplary apparatus for cleaning a nasal cavity, and which can be inserted into the nostril of a user and remove materials therefrom.

FIG. 2 illustrates a side view of an exemplary apparatus for cleaning a nasal cavity, and which has gripping members on a handle of the apparatus.

FIG. 3 illustrates an embodiment of a replaceable and/or disposable nasal cleaning portion of an apparatus for cleaning a nasal cavity, according to another embodiment of the present invention.

FIG. 4 illustrates an alternative embodiment of an exemplary apparatus for cleaning a nasal cavity.

FIG. 5 illustrates is a close-up view of an exemplary nasal cleaning cloth.

FIG. 6 illustrates a close-up view of an alternative exemplary nasal cleaning cloth.

FIG. 7 illustrates a perspective view of a nasal cleaning apparatus according to another embodiment.

FIG. 8A illustrates a partial cross-sectional view taken along section line 8A-8A of the apparatus shown in FIG. 7.

FIG. 8B illustrates a cross-sectional view taken along section line 8B-8B of the apparatus shown in FIG. 7.

FIG. 9 illustrates a perspective view of the apparatus shown in FIG. 7 inserted in a nostril.

FIG. 10 illustrates an apparatus for cleaning a nasal cavity according to another embodiment.

FIG. 11 illustrates a perspective view of a nasal cleaning kit according to an embodiment.

FIG. 12 illustrates a partial cross-sectional view taken along section line 12-12 of the kit shown in FIG. 11.

FIG. 13 illustrates an exploded view of the kit shown in FIG. 11 after the kit is opened.

FIG. 14 illustrates an exploded view of nasal cleaning kit according to another embodiment.

### DETAILED DESCRIPTION

Exemplary embodiments of the invention relate to apparatus and assemblies for cleaning nasal cavities. In particular, exemplary embodiments of the present invention relate to a nasal cleaning apparatus which may be reusable and/or disposable, or which may have reusable and/or disposable components.

Reference will now be made to the drawings to describe various aspects of exemplary embodiments of the invention. It is understood that the drawings are diagrammatic and schematic representations of such exemplary embodiments, and are not limiting of the present invention, nor are they necessarily drawn to scale. The drawings thus provide one scale; however, no inference should therefore be drawn from the drawings that any dimension of the invention or element is required. In the following description, numerous specific details are set forth in order to provide a thorough understanding of the example embodiments of present invention. It will be obvious, however, to one of ordinary skill in the art that the present invention may be practiced without these specific details.

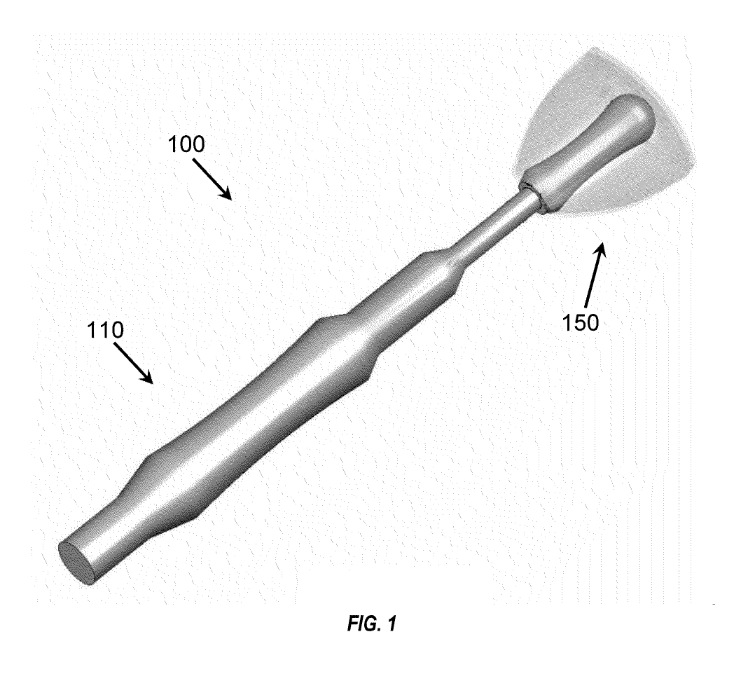

Referring now to FIG. 1, an exemplary apparatus 100 for cleaning a nasal cavity is illustrated according to some aspects of the present invention. In FIG. 1, for example, apparatus 100 includes a grip portion 110 and a cleaning portion 150. Any suitable grip portion 110 and/or cleaning portion 150 may be used. By way of example and not limitation, grip portion 110 may thus include a handle, or other mechanism for gripping or handling apparatus 100, while cleaning portion 150 may be, or include, a swab, cleaning cloth, head, or other type of portion used for cleaning the inside of a nasal cavity, and/or may be a combination of different components.

The embodiment illustrated in FIG. 1 is an example of a suitable apparatus 100 for cleaning inside of a user's nose. For example, cleaning portion 150 may extend from grip portion 110 and be used to clean the interior surfaces of the nostrils to keep them clean and free of dust, nasal discharge, mucous, waste, debris, or any other particles or organisms which may become lodged therein.

In the particular embodiment illustrated in FIG. 1, cleaning portion 150 and grip portion 110 are connected and operate together for cleaning of the inside of the user's nostrils. For example, a user can grab a hold of grip portion 110 with the user's hand, fingers, and/or finger tips, and then extend cleaning portion 110 into the user's own nostril, or into the nostril of another. When inside the nostril, cleaning portion 110 can be moved around and/or rotated so as to contact and collect debris and other materials therein. For example, cleaning portion 110 can be rotated as it is placed against the walls of the nasal cavity to clean off any debris lodged against such walls.

In the illustrated embodiment, the tip of cleaning portion 150 has a generally rounded configuration. One feature of such a configuration is that the rounded taper on the tip can allow cleaning portion 110 to safely and comfortably be inserted into the nostril, and it can then be rotated within the nostril to more effectively reach and clean the nasal cavity. According to one embodiment, cleaning portion 150 is configured to rotate by manual means (e.g., the user manually rotating cleaning portion 150 or apparatus 100), or by electrical or mechanical means. For instance, apparatus 100 may include an electric adapter or battery and a motor (not shown) which causes cleaning portion 150 to rotate. In this manner, by turning on the motor, the tip of cleaning portion 150 can automatically rotate within the nostril to effectively clean the nostril and remove debris therefrom. Alternatively, the motor can cause cleaning portion 150 to vibrate or to vibrate and rotate.

In another embodiment, apparatus 100 does not have a motor or other mechanical or electrical means for rotation. Instead, the user can rotate grip portion 110 in his/her hand or fingers to thereby also rotate apparatus 100. In still another embodiment, a non-motorized nasal cleaning apparatus can include manual rotation means therein which can be powered by the user to rotate cleaning portion 150 without rotating grip portion 110. For instance, a rotary track wheel (not shown) may be included within, or adjacent to, grip portion 110 which can be rotated by the user's fingers. The track wheel can then be connected to gearing or other linkages which link the track wheel to cleaning portion 150, thereby causing cleaning portion 150 to rotate as the track wheel is rotated. As another example, a manual rotation means may include a wind-up feature with a resilient member that stores energy. When that stored energy is released (e.g., by pressing a button), the energy can be used to rotate cleaning portion 150.

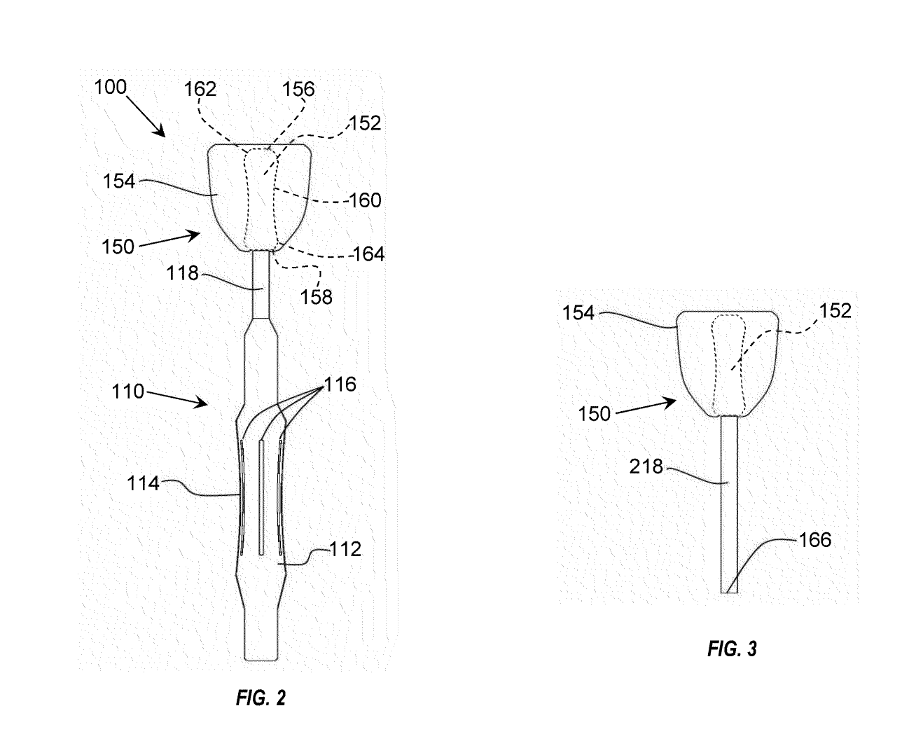

Turning now to FIG. 2, it can be seen that grip portion 110 and cleaning portion 150 can include a variety of optional components. In the illustrated embodiment, for instance, grip portion 110 includes a handle 112 that where user holds onto apparatus 100, and which allows the user to grasp, handle, manipulate, and/or maneuver apparatus 100. Handle 112 may have any suitable shape and configuration that provides the user with such ability. For example in the illustrated embodiment, handle 112 is contoured and has a concave contour 114 along the outer perimeter of all or a portion of handle 112. Concave contour 114 is ergonomically formed to provide a mechanism by which the user can comfortably grasp, handle, and use apparatus 100. Of course, in other embodiments, other types of contours or other ergonomically desirable forms may be utilized, or no contour or other ergonomically desired shape is used. For instance, in some embodiments, bumps and/or depressions may be provided, and any or all of such structures may facilitate manipulation and handling of apparatus 110 by a hand, finger, and/or finger tip of a user.

In one embodiment, handle 112 includes one or more gripping members 116. Gripping members 116 may have any desirable shape or form, and can provide any of a number of different features. For example, according to one embodiment, gripping members 116 help to prevent slippage of a user's fingers or finger tips while the user is handling apparatus 100. Thus, gripping members 116 can help to create a non-slip or substantially non-slip surface on handle 112. Additionally, gripping members 116 can allow the user to more easily rotate or otherwise manipulate apparatus 100 in a desired manner. For example, as discussed herein, it may be desirable to rotate grip portion 110 so as to cause cleaning portion 150 to rotate as it cleans debris or other materials from the inside of a person's nostril. Gripping members 116 may provide a tactile surface on which the user can create pressure with his or her fingers, thereby more easily manipulating and rotating apparatus 100.

Gripping members 116 may have any suitable design, form and/or construction. For example, in one embodiment, gripping members 116 include one or more ridges extending along at least a portion of the elongate length of handle 112. Such ridges may be integrally formed or may be formed separately and affixed to handle 112 with an adhesive, or may even be formed on a sleeve or other component that is thereafter attached around handle 116. Furthermore, any number of such ridges or gripping members 116 may be utilized. In FIG. 2, for example, three gripping members 116 are illustrated on one side of handle 114. As will be appreciated, another three gripping members 116 may also be provided on the opposite side of handle 114, although such number of gripping members is merely exemplary, and more or fewer gripping members 116 may be provided.

Gripping members 116 may also take other shapes and forms. For example, in one embodiment, a gripping member may merely take the form of the concave contour 114. In other embodiments, one or more bumps, depressions, channels, circumferential or orthogonal ridges, or any combination thereof may be utilized as a gripping member 116 to facilitate handling, manipulation and/or use of apparatus 100. Gripping members 116 may also take the form of a non-slip surface, such as a sleeve made of rubber or another non-slip material that is then attached to handle 114.

Handle 116 allows the user to grasp and use apparatus 100, and may have any desirable size and shape, and may further be connected directly or indirectly to cleaning portion 150. In the embodiment illustrated in FIG. 2, grip portion 110 includes an extension member 118 that connects to, and extends from, handle 112 and connects to cleaning portion 150. Extension member 118 can act as a neck that facilitates attachment between handle 114 and cleaning portion 150, and it will be appreciated in view of the disclosure herein that extension member 118 may be integrally formed with handle 114, or may be formed separate therefrom. Indeed, in one embodiment, all or a portion of extension member 118 may be selectively detachable from handle 112 and/or cleaning portion 150.

Extension member 118 may also have any desirable length, shape, or form. According to one aspect, extension member 118 may have a substantially uniform cross-section and/or may have a length calculated to allow the user of apparatus 100 to grasp handle 114 and easily and comfortably place cleaning portion 150 into the user's nostril. For instance, when a user places his or her fingers on handle 114 (e.g., at gripping members 116), extension member displaces cleaning portion 150 from the user's fingers so that the user can easily and comfortable insert cleaning portion 150 into the user's nose and clean the nasal cavities therein. In one example, handle 114 may be approximately two and one-half inches in length, and extension member 118 may extend approximately seven-eighths inch. Not all of extension member 118 may be visible, however, and a portion of extension member 118 may extend into cleaning portion 150. Alternatively, extension member 118 may connect directly to cleaning portion 150 without extending therein and/or may have a variable cross-section across its length.

With continued reference to FIG. 2, apparatus 100 may also include a cleaning portion 150 that is directly or indirectly connected to grip portion 110. In the illustrated embodiment, cleaning portion 150 is constructed and adapted to provide a sanitary and hygienic mechanism for cleaning debris or other materials from the inside of the user's nostril. Cleaning portion 150 may have any suitable construction, design, and form, accordingly the embodiments illustrated and described herein are merely embodiments and are not necessarily limiting of the present invention.

In the embodiment illustrated in FIG. 2, cleaning portion 150 includes a head 152 and a nasal cleaning cloth 154. In particular, head 152 is connected to extension 118 of grip portion 110, and nasal cleaning cloth 154 is wrapped or otherwise placed around the exterior of head 152. Head 152 thus provides an internal structure that supports nasal cleaning cloth 154 as nasal cleaning cloth 154 is inserted into the nose of a user and is used to clean debris, pollen, dust, and/or other materials therefrom.

With more particular attention to the exemplary embodiment illustrated in FIG. 2, head 152 is illustrated in phantom lines to depict that it is positioned inside of nasal cleaning cloth 154. It should be appreciated in view of the disclosure herein that such is not always necessary. For example, in one embodiment, head 152 may be integrally formed with nasal cleaning cloth 154, such that the outer surface of head 152 is used to clean the inside of a user's nostril. Accordingly, nasal cleaning cloth 154 may be a material that is separate from, but attached to, head 152, or it may be integral therewith.

Head 152 may be designed as a support member that facilitates attachment of nasal cleaning cloth 154 to grip portion 152, and may also support nasal cleaning cloth 154 while it is within the nostril of a user. Head 152 may thus have any suitable shape or form that provides these or other features. Head 152 may further be shaped in any manner that allows it to be inserted into the nostril of a user, and may have a shape that facilitates its use with a wide variety of nostril sizes and shapes. In the illustrated embodiment, for instance, a contoured and tapered configuration is illustrated that includes a plurality of different contour portions. For example, in the illustrated embodiment, head 152 includes a middle portion having a substantially concave portion 160. As illustrated in FIG. 2, for example, concave portion 160 includes an inwardly curving surface. On either side of concave portion 160, such as at distal end 156 and proximal end 164, there may be convex portions 162, 164. For instance, at distal end 156, convex portion 162 may be have a generally semi-spherical shape. The semi-spherical shape tapers inward toward distal end 156. Distal end 156 is the first portion of apparatus 100 that is inserted into a user's nose. Thus, by tapering outward from distal end 156, head 152 facilitates easy insertion and advancement of apparatus 100 at least part way into the user's nostril. Head 152 need not always have such a construction, however and may have any number of different shapes, forms and/or contours.

As noted previously, the shape of head 152 is merely one example of a head usable as all or a part of cleaning portion 150, and head 152 may have any other desired shape or form. For example, in one embodiment, head 152 may have only a single contoured portion. For instance, head 152 may be generally spherical or balloon-shaped such that substantially all surfaces are convex. Alternatively, head 152 may have only one, or more than one, concave surface, may have non-contoured and/or tapered portions, may have any combination of the above, or any other suitable shape.

To further facilitate cleaning and removal of debris from the inside of the nostril of a user, a nasal cleaning cloth 154 may be attached to head 152. Nasal cleaning cloth 154 is, in the illustrated embodiment attached to the exterior of head 152. Nasal cleaning cloth 154 may also be a pliable material that can be shaped as desired by the user. Thus, apparatus may be produced such that the shape of nasal cleaning cloth 154 essentially conforms to the shape of head 152, although it is not necessary to do so. In some embodiments, nasal cleaning cloth 154 may be a more rigid material that is formed around, or slipped onto, head 152, or it may have any other suitable form. In one example, for instance, nasal cleaning cloth 154 is a flexible material that is formed separate from head 152, and is thereafter wrapped thereon.

In one method for manufacturing apparatus 100, nasal cleaning cloth 154 may be initially cut or otherwise formed into a square or diamond shaped piece of cloth. To attach nasal cleaning cloth 154 to head 152, nasal cleaning cloth 154 may first be folded along a diagonal to form a triangular shape. Head 152 may be placed on the interior of nasal cleaning cloth 154, and may be positioned proximate the fold line. With head 152 in that position, cloth 154 can be wrapped, folded, or draped around head 152 and the apex of the formed triangle can be secured to proximal end 158 of head 152. In one example embodiment, head 152 has an opening (not shown) at proximal end 158, and portions of nasal cleaning cloth 154 can be tucked into the opening. Grip portion 110 may then be placed in the opening of head 152 to further secure nasal cleaning cloth 154 therein. In addition, or in the alternative, an adhesive may also be used to secure nasal cleaning cloth 154 to head 152 at proximal end 158 and/or at distal end 156, or at portions between distal end 156 and proximal end 158. An adhesive or other attachment mechanism may also be used to secure head 152 to extension 118 or to grip portion 110.

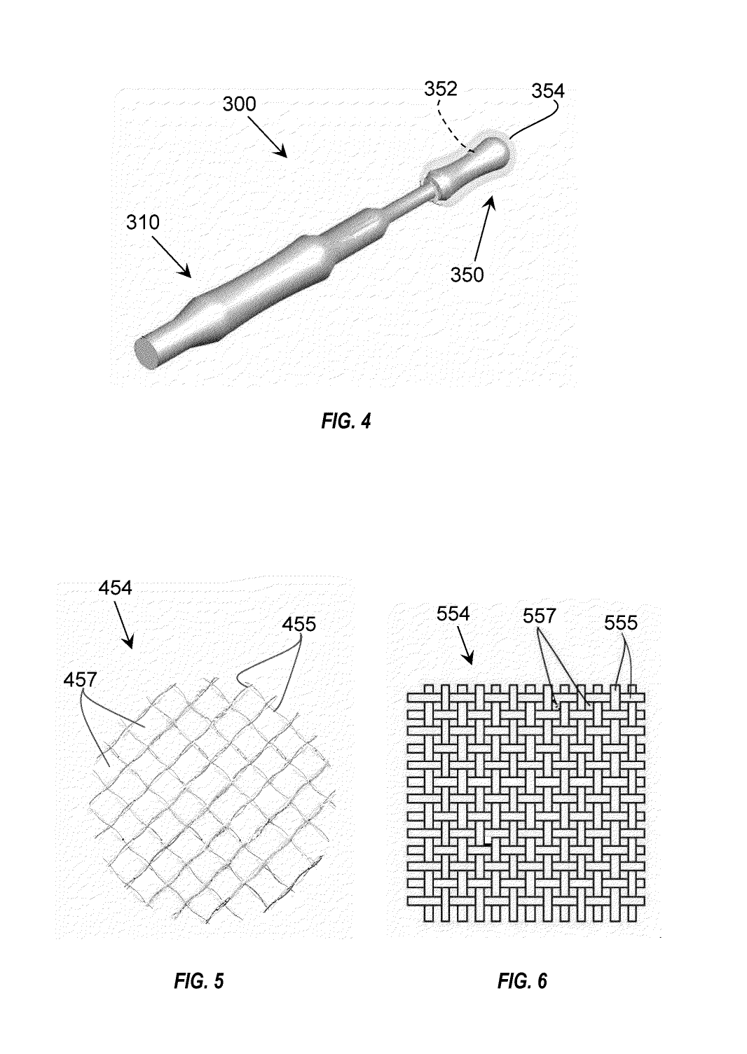

Securing nasal cleaning cloth 154 in this manner or any other suitable manner can allow nasal cleaning cloth 154 to be generally formed such that it is supported by head 152, and optionally has a shape that generally conforms to that of head 152, although it is not necessary to do so. For instance, when the apex of the triangle formed by folding nasal cleaning cloth 154 is secured to proximal end 158 of head 152, the triangles may form around head 152 in a manner that does not necessarily conform to the shape of head 152. FIGS. 1-3, for example, illustrates a nasal cleaning cloth 154 that is placed around a head 152, but does not necessarily conform to the shape of head 152. Alternatively, FIG. 4 illustrates a nasal cleaning cloth 354 which does substantially conform to the shape of head 352. It will be appreciated in view of the disclosure herein, that the shapes of a nasal cleaning cloth as described herein—both before and after attachment to a head or other component of a nasal cleaning apparatus—are merely exemplary and that other shapes, forms, and attachment mechanisms or methods are contemplated within the scope of the present invention.

The various components of a nasal cleaning apparatus 100 according to the present invention can be made from any of numerous different materials, and can be formed in any suitable manner. For example, nasal cleaning cloth 154 can be formed from any suitable material that would allow and/or facilitate removal of debris and other material from inside a nasal cavity. In one example, nasal cleaning cloth 154 is formed from an anti-microbial material and/or a sterilized material. One feature of such materials is that they can be placed into the nose and clean out materials therein, with reduced risk of introducing additional infective materials or organisms that might cause allergies, the common cold, or the flue, thereby reducing the effectiveness thereof. Any suitable anti-microbial or sterilized material may be utilized. For instance, a microfiber anti-microbial material may be used. In another embodiment, a nasal cleaning cloth 154 may be a fabric which is resistant to microbes and/or bacteria, or which has been treated with an anti-microbial compound. Additionally, or in the alternative, nasal cleaning cloth 154 can be medicated to allow medicine to be applied to the nasal cavity.

In one embodiment, nasal cleaning cloth 154 is made of a soft material so as to gently adhere to material in the nostrils and to safely clean the nostrils. In another embodiment, nasal cleaning cloth 154 is made of a flexible material which is at least partially rough and/or has pores or cavities formed therein (e.g., a mesh material). Such a material may allow nasal cleaning cloth 154 to rub against the interior surface of the nasal cavity and more easily dislodge debris attached thereto, and the pores or cavities may provide space within nasal cleaning cloth 154 to collect and hold such debris. In another embodiment, the tip is made of a rigid material which will effectively rub against materials in the nostrils and dislodge them. Optionally, materials of differing levels of rigidity are used to both dislodge materials and to cause materials to adhere to the cleaning portion. In short, any material, or combination of materials, may be used which, when inserted into the nostril, will help remove and eliminate germs, bacteria, dust, pollen, viruses, mucous, or any other type of substance of any kind which may be found within the nose, nostrils, or nasal cavities and which would be beneficial to remove. Such material may also be configured to be washed for repeated uses. For example, after a use, a user may run cleaning portion 150 under water to clean nasal cleaning cloth 154 so that it can be used on other occasions. In one embodiment, cleaning portion, including nasal cleaning cloth 154, is adapted to be cleaned and reused. For example, nasal cleaning cloth 154 may be made of a material (e.g., polymeric material, organic-based material, etc.) that can hold up under cleaning as it is washed with, for example, a soft soap and warm water.

Head 152 and grip portion 110 may also be formed of any suitable material. In one example, head 152 is formed of a material which sufficient rigidity to provide support nasal cleaning cloth 154 as cleaning portion 150 is inserted into a nostril and engages the interior surface of the nostrils to clean debris and other materials therefrom. For example, a foam material may be used. Such a material may be sufficiently rigid to support nasal cleaning cloth 154 and to provide a surface around which nasal cleaning cloth 154 can be positioned or formed, but may also be somewhat pliable such that it yields and does not easily damage tissue within the nasal cavity. Of course, other materials may also be used, including polymers, rubber, composites, organic materials, metals, alloys, or other materials or combinations thereof. Handle 114 and/or extension 118 may also be formed of similar materials. For instance, handle 114 and extension 118 may be integrally formed of a substantially rigid polymeric material. Alternatively, handle 114 and extension 118 may be separately formed and/or be made from a foam, rubber, composite, organic, metal, alloy, or other material, or a combination thereof.

As further evident at least the embodiments depicted in, and described relative to, FIG. 2 and FIG. 3, in some embodiments a nasal cleaning apparatus 100 may be fully or partially reusable and/or disposable. For instance, FIG. 2 illustrates an example apparatus 100 which may be fully disposable. In particular, apparatus 100 may be designed and constructed to for a single use or for multiple uses, after which it should be discarded in its entirety. Alternatively, the embodiment illustrated in FIG. 3 shows an example portion of apparatus 100 that may be utilized when only a portion of apparatus 100 is intended to be disposable.

For instance, in the embodiment illustrated in FIG. 3, a cleaning portion 150 that includes an extension 218. In this embodiment, extension 218 may be selectively removable from handle 114 (FIG. 2). This may allow, for example, handle 114 to be reused with any number of different cleaning portions 150, and each disposable cleaning portion 150 can be discarded after one or more uses.

To facilitate selective detachment with handle 114, cleaning portion 150 may have a mechanism for providing selective attachment to, and detachment from, handle 114. For example, threads (not shown) may be formed on a proximal end 166 of extension 218 and configured to mate with corresponding threads found in handle 114. It will be appreciated, however, that any other suitable connection mechanism may be used, including lock fits, interference fits, clasps, clamps, or any other connection mechanism or combination thereof.

Furthermore, extension 218 may itself also be reusable apart from head 152 of cleaning portion 150. For example, it may also be possible to remove head 152 from extension portion 218 in the event that extension 218 or head 152 becomes damaged or worn. Thus, a nasal cleaning apparatus according to the present invention can have a modular construction allowing any of numerous components to be selectively attached and detached as desired.

It will be appreciated that one feature of removable and/or replaceable components is that the same components (e.g., handle, extension, head, etc.) may be used multiple times to safely and efficiently clean the nostrils. This in turn reduces the cost of the product inasmuch as an entirely new product is not needed for each subsequent use. Instead, in embodiments having a disposable head, only the head and/or nasal cleaning cloth need be obtained for each use or for limited uses. It will also be appreciated that by having a reusable handle or grip that is not replaced each time, the grip can be cost-effectively engineered for comfort and suitability for continued use. For instance, as discussed above, the handle may be engineered to have a mechanical and/or electrical system for rotating the cleaning portion. The grip may also be made of soft-grip materials to increase the comfort of use and/or contoured for a comfortable grip.

FIG. 4 illustrates another embodiment of an example nasal cleaning apparatus 300 similar to that in FIG. 1, but having a nasal cleaning cloth 354 of a different shape. In particular, in the embodiment illustrated in FIG. 4, nasal cleaning cloth 354 is attached to, or otherwise substantially encompasses, head 352. Whereas FIG. 1 illustrates a nasal cleaning cloth that does not necessarily conform to the full shape of the head of the cleaning portion, FIG. 4 illustrates that nasal cleaning cloth 354 can be made to conform generally to the shape of head 352. In this embodiment, nasal cleaning cloth 354 is illustrated as partially transparent so as to show head 352, and nasal cleaning cloth 354 is shaped to have concave and convex sections generally corresponding to concave and convex curves on head 352. To conform to the shape of head 352, it is not necessary, however, that all contours of head 352 be matched by nasal cleaning cloth 354. For example, in the illustrated embodiment, the concave curve towards the middle of nasal cleaning cloth 354 could be less concave, could not taper at all, or could be convex, and nasal cleaning cloth 354 may still substantially conform to the shape of head 352.

As will also be appreciated in view of the disclosure herein, while FIG. 4 illustrates an embodiment similar to that of FIG. 1, head 352 and/or nasal cleaning cloth 354 may equally be used with a modular or removable apparatus such as that illustrated in FIG. 3. Accordingly, components, structures, and materials of any embodiment herein may be combined with the other disclosed embodiments.

In view of the foregoing, it will be appreciated that a nasal cleaning apparatus of the present invention may be manufactured or produced to have any of a variety of different sizes and configurations. In one example, the grip portion of the apparatus may have a total length of approximately three and one-third inches, and a maximum width of approximately three-eighths inches. Additionally, the head of the cleaning portion can have a length of approximately three-quarter inches and a maximum diameter of about one-quarter inch. At this size, the head can easily be inserted into a nostril of a typical user with tolerances to allow the user to rotate the cleaning portion as needed to clean the debris, dust, mucosal buildup, bacteria, viruses, pollen, waste, discharge, or any other materials therefrom. The nasal cleaning cloth 154 that is attached to the head may also have any desirable size. For instance, in one embodiment, the nasal cleaning cloth is formed of an anti-microbial material that is diamond-shaped and approximately one and one-half inches along each side. This allows the nasal cleaning cloth to be folded around the head while still providing sufficient cleaning material to ensure an effective cleaning of the interior of a user's nostrils.

Turning now to FIGS. 5 and 6, close-up views of exemplary materials that may be used in connection with at least the nasal cleaning cloth are illustrated. In FIG. 5, for example, an exemplary cloth 454 is illustrated that has an open-mesh type construction. In particular, this exemplary material includes a variety of material strands 455 that are formed into a weaved pattern. As further shown, the weaved pattern creates various pockets 457. In operation, strands 455 of cloth 454 can be engage against materials, skin, and tissue within a user's nose and cause debris to be removed therefrom. The debris may then adhere to strands 455 and/or be captured within pockets 457. As can thus be seen, cloth 454 may thus have a weaved and/or mesh type pattern.

The size, construction, and material of strands 455, and the size and construction of pockets 457 can be varied as desired. In the illustrated example, for instance, strands 455 can be formed of more than one thread. Moreover, while the threads of strands 455 are generally illustrated as following a defined pattern, the threads may also extend in many different directions. For instance, threads forming strands 455 can extend randomly, or pseudo-randomly to form. On feature of such type of threads is that strands 455 can thus produce an exfoliating effect. In particular, the random or untamed threads can contact against the interior of the nose and more effectively brush away dead skin, debris, germs, or other materials. The effect may thus be similar to an exfoliating loufa.

To further the exfoliating effect, strands 455 may be formed of a durable material (e.g., a plastic). In some cases, some threads are formed of one material (e.g., a durable or hard material), while other threads are formed from a relatively softer material. By way of example, suitable materials that can be used to form durable and/or softer materials within strands 455, include polymer-based materials (nylons, polyamides, polypropylene, etc.), organic materials (e.g., cotton, wool, etc.) and or any combinations thereof or of any other suitable material. For example, in one embodiment, cloth 454 is an antimicrobial, exfoliating material in which strands 455 are formed of a polyamide-polypropylene combination (approximately 73% polyamide and 27% polypropylene).

The size of strands 455 and/or pockets 457 can also vary in a desired manner. In one example, pockets 457 bordered by strands 455 can be approximately one-sixteenth inch to one-quarter inch in width, although pockets may also have smaller or larger sizes, as desired. For example, pockets may have a square or diamond shape that has sides that are approximately one-eighth inch in length, although pockets may have other shapes, sizes, and configurations. Strands 455 may also be various sizes. For example, strands 455, whether made of one or more threads, may, in one embodiment, be between one-thirty second inch and three-thirty seconds inch in width or diameter, although they may have a greater or smaller width or diameter if desired.

Turning now to FIG. 6, another example embodiment of a fabric material is schematically illustrated. In FIG. 6, a cloth 554 is illustrated that also includes a plurality of strands 555 which are woven together to also form a plurality of pockets 557. As can be seen when compared with cloth 454 in FIG. 5, cloth 554 of FIG. 6 has a tighter weave or tighter strand 555 pattern. For example, in this embodiment, strands 555 and pockets 557 have approximately the same width, although this is exemplary only. Moreover, while pockets 557 are shown as having a generally square shape, this isn't necessary, and pockets 557 can be circular, rectangular, or have any other desired shape. For instance, if every other strand 555 along the vertical distance is removed, pockets 557 may have a generally rectangular shape.

As with other nasal cleaning cloths described herein, cloth 554 may also be formed of any suitable material, and strands 555 may have any suitable size. Further, it is not necessary to have strands 555, as cloth 554 need not have a woven construction, but may be otherwise formed. Accordingly, it will be appreciated that cloth 554 may have any suitable design. For example, strands 555 may be sized on the orders of microns or may be larger (e.g., about a quarter inch in width), or anywhere in between. Where desired, strands 555 may also have larger or smaller sizes or, as noted above, strands 555 may be excluded entirely.

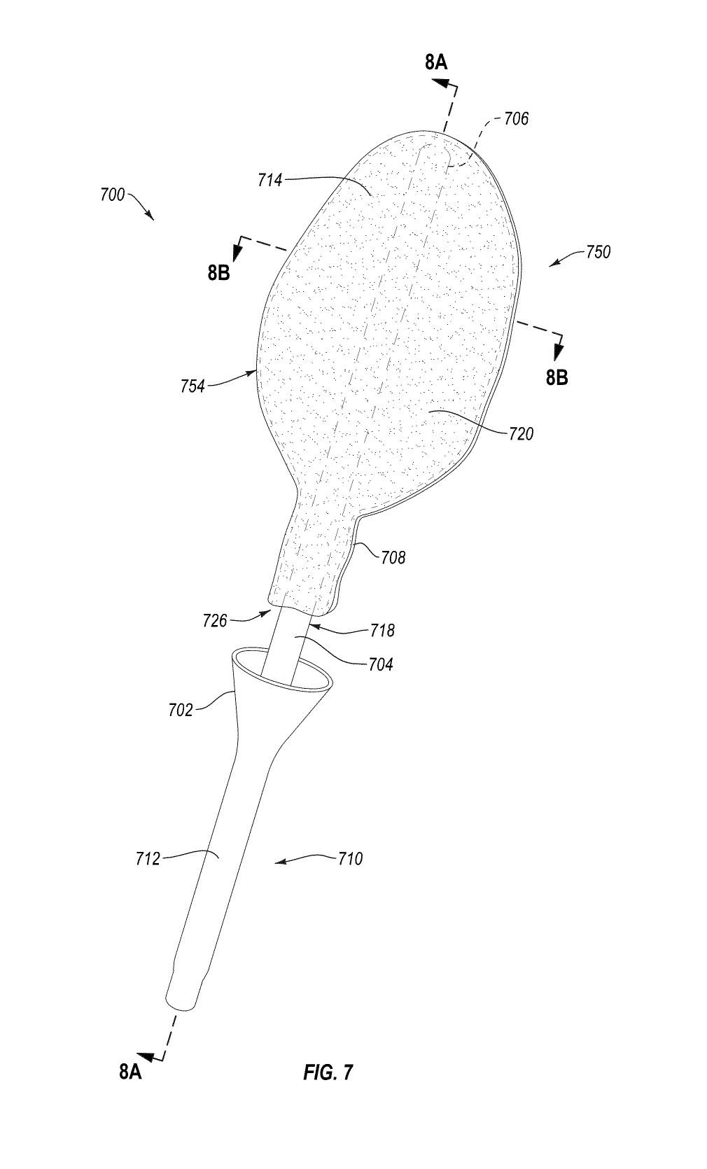

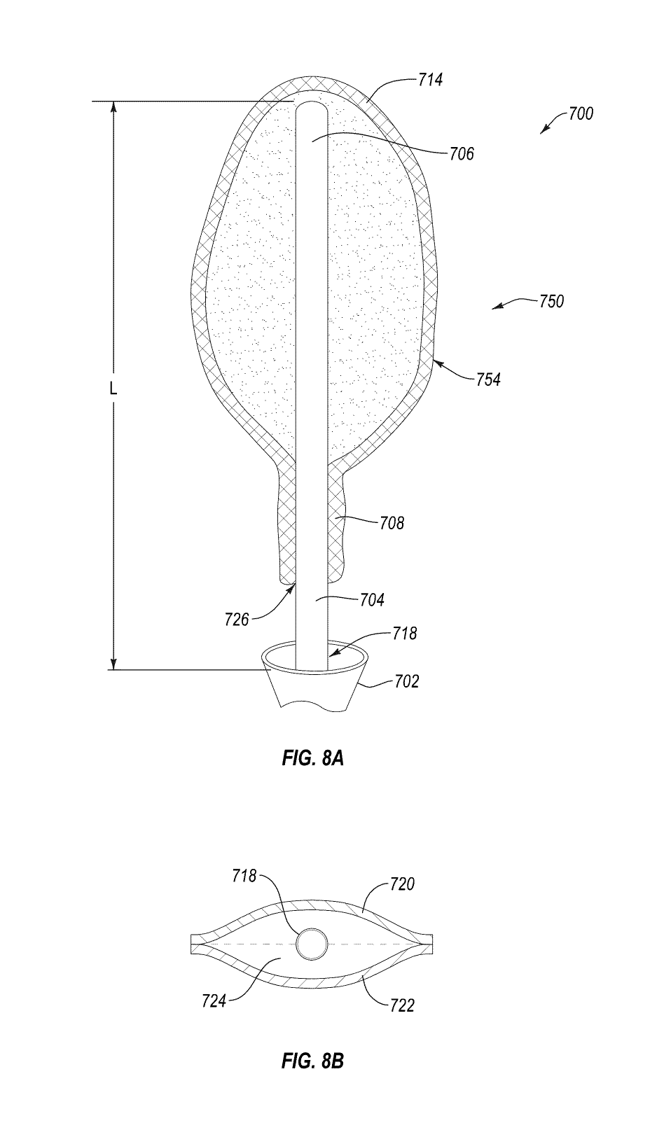

Referring now to FIGS. 7, 8A, and 8B, a nasal cleaning apparatus 700 will be described according to another embodiment. The nasal cleaning apparatus 700 may be similar in many respects to the apparatus 100 and/or apparatus 300 previously described above in relation to FIGS. 1 through 6. Therefore, in the interest of brevity, the components and features of apparatus 700 and apparatuses 100 and 300 that correspond to each other have been provided with the same or similar reference numerals, and an explanation thereof will not be repeated. However, it should be noted that the principles of the nasal cleaning apparatus 700 may be employed with any of the embodiments described in relation to FIGS. 1 through 6 and/or vice versa.

Apparatus 700 may include a grip portion 710 and a cleaning portion 750. In the illustrated embodiment, the grip portion 710 may include a handle 712 configured to allow a user to hold, grasp, handle, manipulate, and/or maneuver nasal cleaning apparatus 700. Handle 712 may have any suitable shape and/or configuration. For example, in an embodiment, handle 712 may include a generally conical distal portion 702 that reduces in diameter and cross-sectional area as the conical distal portion 702 extends proximally toward a body portion of the handle 712. The conical distal portion 702 may be configured as a hollow conical portion or a solid conical portion. For example, a hollow conical portion may be configured to help capture debris and/or other materials that may escape from a nostril onto the grip portion 710.

The body portion of the handle 712 may exhibit a generally cylindrical cross-sectional shape. In other embodiments, the body portion of the handle may exhibit a generally elliptical cross-sectional shape, a generally rectangular cross-sectional shape, an irregular geometric cross-sectional shape, combinations thereof, or any other suitable geometric shape. The handle 712 may also be solid or may exhibit a generally tubular configuration.

The handle 712 may be ergonomically formed or shaped to help provide a mechanism by which a user can comfortably grasp, handle, and/or use apparatus 700. In other embodiments, other types of shapes or other ergonomically shaped forms may be used, or no contour may be used. For instance, in some embodiments, the handle 712 may include raised portions and/or depressions to enhance a user's grip on the handle 712 and/or handling of the apparatus 700. The handle 712 may be formed of any suitable material. For example, handle 712 may include one or more materials with sufficient rigidity to support at least cleaning portion 750. In an embodiment, handle 712 may include one or more plastic materials, polymeric materials, organic-based materials, rubber materials, polyethylene materials, metals, metal alloys, fabric materials, combinations thereof, or the like.

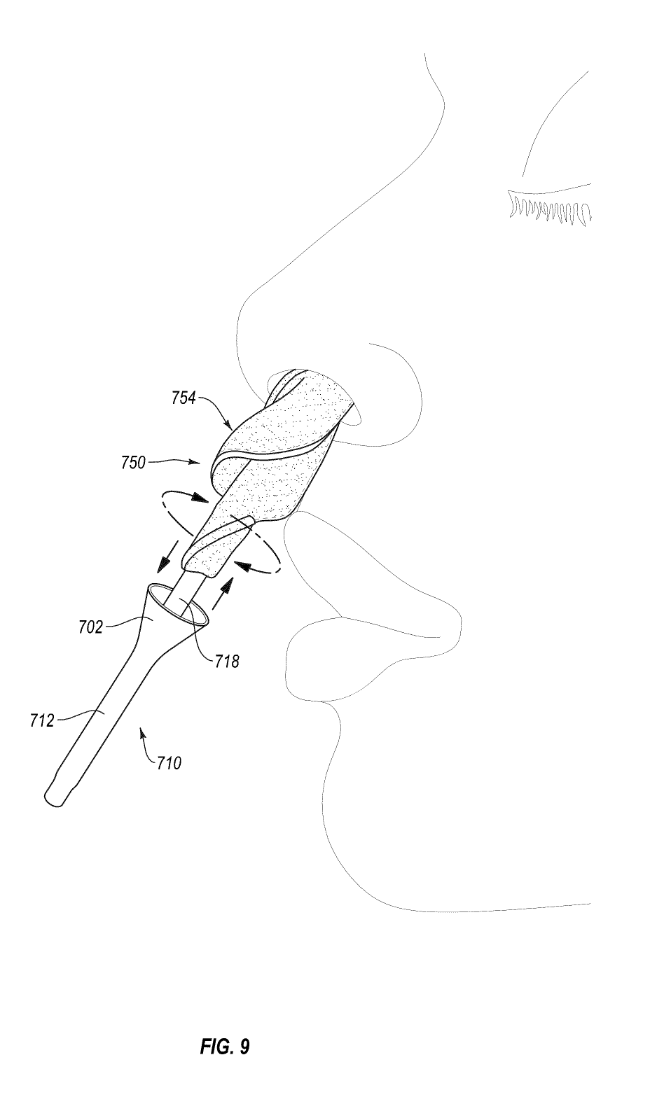

While the handle 712 is illustrated including a conical proximal portion, in other embodiments, the proximal portion of the handle 712 may be generally cylindrical, generally rectangular, generally elliptical, or may have any other suitable shape. Moreover, while the handle 712 is illustrated including a cross-sectional area that varies, in other embodiments, the cross-sectional area of the handle 712 may be generally constant or may vary in one or more different locations. In yet other embodiments, the conical distal portion 702 of the handle 712 may be omitted. For example, the handle 712 may include a constant cylindrical configuration. Moreover, in other embodiments, handle 712 may include a shield member (shown in FIG. 9) configured to help limit saline solution, body fluids, or other fluids or materials from running down from the cleaning portion 750 onto a user's hand. For example, handle 712 may include an annular member attached to the proximal portion of the handle 712. The annular member may have an outer diameter between about two (2) and five (5) times greater than a diameter of the cylindrical body of the handle 712. In other embodiments, the annular member may have an outer diameter larger or smaller relative to the diameter of the cylindrical body of the handle 712. Such a configuration may help catch and/or divert fluids from running onto a user's hand during use. In other embodiments, the shield member may be configured to help prevent over insertion of the nasal cleaning apparatus 700 into the user's nostril. In other embodiments, the handle 712 may include one or more gripping members (shown in FIG. 2) configured to prevent slippage of a user's fingers or finger tips while the user is handling apparatus 700. Moreover, gripping members may allow a user to more easily rotate or otherwise manipulate apparatus 700 in a desired manner.

Grip portion 710 may further include an extension member 718 extending from handle 712. More specifically, extension member 718 may include a proximal end portion 704 connected to handle 712 and a distal end portion 706 positioned within the cleaning portion 750. Extension member 718 may be integrally formed with handle 712 or may be attached to handle 712. For example, in an embodiment, the handle 712 may include an internal lumen (not shown) extending between a proximal end of the handle 712 and the distal end portion 702. In other embodiments, the lumen of the handle 712 may extend only a portion of the distance between the proximal end and the distal end portion 702 of the handle 712. The handle lumen may be configured to receive and secure at least a portion of the extension member 718 within the handle 712. For example, a portion of the extension member 718 may be secured within the lumen of the handle via a press-fit connection, adhesives, detents, or by any other suitable means. Such a configuration may help strength the extension member 718 against undesired movement of the extension member 718. In other embodiments, the extension member 718 may be attached to the distal end portion 702 of the handle 712. In yet other embodiments, the extension member 718 may be selectively detachable from handle 712 and/or cleaning portion 750 such that the extension member 718 may be disposable. Extension member 718 may have any suitable, length, size, configuration or form. For example, in an embodiment, extension member 718 may be generally elongated and may have a substantially uniform and generally circular cross-sectional area. Extension member 718 may have a generally solid cross-sectional configuration or extension member 718 have a tubular hollow configuration. Moreover, extension member 718 may have a length L defined between the distal end portion 702 of the handle 712 and the distal end portion 706 of the extension member 718. The length L of extension member 718 may be selected to allow a user to safely and effectively position cleaning portion 750 into the user's nostril. For example, the length L of extension member 718 may be between about one (1) inch and six (6) inches; between about two (2) inches and five (5) inches; or between about three (3) inches and four (4) inches. In other embodiments, the length L of extension member 718 may be longer or shorter. In addition, the extension member 718 may be formed of any of the materials discussed with reference to handle 712. For example, extension member 718 may include one or more materials with sufficient rigidity (e.g., polymeric material, organic-based material, metals) to support cleaning portion 750 being inserted or used in a user's nostril or nasal cavity. In some embodiments, the extension member 718 may be formed of the same materials as the handle 712. In other embodiments, the extension member 718 and handle 712 may include one or more different materials.

While extension member 718 is illustrated having a generally circular cross-sectional shape, in other embodiments, extension member 718 may exhibit a generally elliptical cross-sectional shape, a generally rectangular cross-sectional shape, a generally diamond cross-sectional shape, a generally cruciate cross-sectional shape, combinations thereof, or any other suitable cross-sectional shape. Further, while extension member 718 is illustrated having a generally constant cross-section, in other embodiments, extension member 718 may exhibit a cross-section that varies in diameter and/or shape. For example, in one embodiment, the extension member 718 may include a generally cylindrical portion having a first diameter and a generally spherical portion at the distal end having a second diameter larger than the first diameter.

Cleaning portion 750 may be directly or indirectly connected to grip portion 710. In the illustrated embodiment, cleaning portion 750 may be constructed and configured to provide a sanitary and hygienic mechanism for cleaning debris or other materials from the inside of the user's nostril. Moreover, the cleaning portion 750 may be constructed and configured to help moisten the inside of the user's nostril. Cleaning portion 750 may include any suitable construction, design, and configuration. For example, in the illustrated embodiment, cleaning portion 750 may comprise a nasal cleaning cloth 754. The nasal cleaning cloth 754 may exhibit a generally balloon-like shape including a generally elliptical body portion having a generally semi-rounded distal end portion 714 and a smaller, generally rectangular proximal end portion 708. In other embodiments, the nasal cleaning cloth 754 may exhibit other suitable shapes. For example, the nasal cleaning cloth 754 may exhibit a generally rectangular shape, a generally diamond-like shape, an irregular geometric shape, a pine tree-like geometric shape, a butterfly like shape, a generally helical shape, combinations thereof, or the like.

In the illustrated embodiment, nasal cleaning cloth 754 may comprise a sock-like structure including a front wall 720 extending between the distal end portion 714 and the proximal end portion 708, and a back wall 722. In an embodiment, the back wall 722 may be extending generally parallel to the front wall 720. Both the front wall 720 and the back wall 722 may include an interior surface and an exterior surface. In an embodiment, the front wall 720 and the back wall 722 may be separate pieces of fabric, for example, attached along a portion of the periphery of the walls, 720, 722 such that the interior surfaces of the front wall 720 and back wall 722 form an internal space 724 (shown in FIG. 8B). Another portion of the periphery of the front wall 720 and back wall 722 may form an opening 726 at the proximal end portion 708 of the nasal cleaning cloth 754 in communication with the internal space 724. The opening 726 may be configured to access the internal space 724. It will be appreciated that the periphery of the walls 720, 722 may include any portion of the walls 720, 722 between the center of the walls 720, 722 and the edges of the walls 720, 722, including the edges of the walls 720, 722. For example, the periphery of the walls 720, 722 may include one or more portions of the walls 720, 722 at or in the general area of the edges of the walls 720, 722.

The front wall 720 and the back wall 722 may be connected together in any suitable manner. For example, in an embodiment, the front wall 720 and the back wall 722 may be stitched, sewn, or threaded together with a polyester thread. In other embodiments, the front wall 720 and the back wall 722 may be attached via adhesives, natural fiber threads, mechanical fasteners, combinations thereof, or by any other suitable means. For example, the front wall 720 and the back wall 722 may be sewn or threaded together with polyester thread along one or more lines generally traversing at least a portion of the front wall 720 and back wall 722. In another embodiment, the front wall 720 and the back wall 722 may be attached together along a line extending through the center of the front wall 720 and/or back wall 722.

In other embodiments, the nasal cleaning cloth 754 may comprise a single piece of fabric folded at the distal end portion 714 to define front wall 720 and back wall 722. The front and back walls 720 and 722 may be attached along a side portions of the periphery of the walls 720, 722 so that the fold at the distal end portion 714 and the side portions of the periphery of the walls 720, 722 at least partially define the internal space 724 within the nasal cleaning cloth 754. Another portion of the periphery of the walls 720, 722 at the proximal end portion 708 of the nasal cleaning cloth 754 may form the opening 726. In yet other embodiments, the nasal cleaning cloth 754 may comprise a single member folded along any other portions (i.e., proximal end portion, side portions) to define the front wall and the back wall which may then be connected together to define the internal space 724. In further embodiments, the nasal cleaning cloth 754 may include three members, four members, or any other suitable number of members.

In yet other embodiments, the nasal cleaning cloth 754 may include one or more layers. For example, the nasal cleaning cloth 754 may include an interior liner attached to the interior surfaces of the front wall 720 and/or back wall 722. In other embodiments, the nasal cleaning cloth 754 may include one or more outer layers attached to the exterior surfaces of the front wall 720 and/or the back wall 722. For example, the nasal cleaning cloth 754 may include an outer layer attached to the exterior surface of the front wall 720 or the back wall 722 that includes one or more rigid material to help rub and/or dislodge materials and/or substances in the nostril and/or nasal cavity. In yet other embodiments, the nasal cleaning cloth 754 may include an outer coating of moisturizer, medicine, ointment, antimicrobial substances, combinations thereof, or the like.

Optionally, the internal space 724 of the nasal cleaning cloth 754 may be configured to provide a supply of one or more materials or substances to a user's nostril or nasal cavity. For example, the internal space 724 may be pre-filled, at least partially, with medicine, moisturizer, saline solution, water, antimicrobial substances, powders, combinations thereof, or any other suitable substance or material. In other embodiments, as a user moistens the nasal cleaning cloth 754 with a substance (i.e., water or saline solution), the substance may pass through pores or voids in the nasal cleaning cloth 754 and collect within the internal space 724 such that the nasal cleaning cloth 754 may provide a supply of the substance for later use in the user's nostril. In yet other embodiments, when inserted into the nostril, debris, germs, bacteria, dust, pollen, viruses, mucus, or any other type of substance dislodged from the user's nostril by the nasal cleaning apparatus 700 may pass through pores or cavities in the nasal cleaning cloth 754 and collect in the internal space 724 for easier removal.

While the nasal cleaning cloth 754 is illustrated as a sock-like body, in other embodiments, the nasal cleaning cloth 754 may exhibit different configurations. For example, the nasal cleaning cloth 754 may exhibit any of the configurations described in relation to nasal cleaning cloths 154, 354. By way of another example, the nasal cleaning cloth 754 may comprise front wall 720 and back wall 722. The interior surfaces of each wall may be connected together via adhesives, stitching, combinations thereof, or the like. In an embodiment, a channel (not shown) may be formed between the walls 720, 722 that extends between the opening 726 and the distal end portion 714 of the nasal cleaning cloth 754. The channel may be sized and configured to receive at least a portion of the extension member 718. In yet other embodiments, the nasal cleaning cloth 754 may comprise a single member have a thickness defined between an interior and exterior surface. For example, the nasal cleaning cloth 754 may comprise a single polyurethane foam member having a channel (not shown) formed therein configured and sized to receive at least a portion of the extension member 718.

As discussed above, the nasal cleaning cloth 754 may include the opening 726 in the proximal end portion. In an embodiment, the opening 726 may be in communication with the internal space 724 such that the extension member 718 of grip portion 710 may be at least partially extended through opening 726 into the internal space 724. The percentage of the length L of the extension member 718 positioned within the internal space 724 of the nasal cleaning cloth 754 may influence the level of support provided by the extension member 718 to the nasal cleaning cloth 754 during use. For example, more than about fifty percent (50%) of the length L of the extension member 718 may be positioned within the internal space 724 of the nasal cleaning cloth 754. In other embodiments, at least about twenty percent (20%), at least about forty percent (40%), at least about sixty percent (60%), at least about eighty percent (80%) of the length L of said extension member 718 may be positioned within the internal space 724 of the nasal cleaning cloth 754. In other embodiments, between about twenty percent (20%) and one-hundred percent (100%); about thirty percent (30%) and ninety percent (90%); forty percent (40%) and eighty percent (80%); and fifty percent (50%) and seventy percent (70%) of the length L of the extension member 718 may be positioned within the internal space 724 of the nasal cleaning cloth 754. In other embodiments, more or less of the length L of the extension member 718 may be positioned within the internal space 724 of the nasal cleaning cloth 754. In other embodiments, the extension member 718 may include one or more perforations. For example, in an embodiment, the handle 712 and/or the extension member 712 may include a lumen extending therethrough and a port in communication with the lumen. Moreover, the length L of the extension member 718 positioned within the nasal cleaning cloth 754 may include one or more perforations also in communication with the lumen. Accordingly, medicine, ointment, moisturizer and/or other substances may be placed in the lumen via the port such that the substances may be dispensed through the perforations into the interior space of the nasal cleaning cloth 754 during use. In other embodiments, the opening 726 may be formed in other portions of the nasal cleaning cloth 754. For example, the opening 726 may be formed in the elliptical body portion of the nasal cleaning cloth 754, the distal end portion, or any other suitable portion of the nasal cleaning cloth 754.

The nasal cleaning cloth 754 may be connected to the extension member 718 in a variety of different configurations. For example, in the illustrated embodiment, the proximal end portion of the nasal cleaning cloth 754 may be attached to the extension member 718 via one or more medical grade adhesives while the distal end portion of the nasal cleaning cloth 754 and a portion of the front wall 720 and the back wall 722 are not attached to the extension member 718. Such a configuration may allow the distal end portion 714 and the unattached portions of the front wall 720 and the back wall 722 to be rotatable and/or movable relative to the proximal end portion 708 of the nasal cleaning cloth 754 and/or the extension member 718. Accordingly, a user may twist the distal end portion 714 and the unattached portions of the front wall 720 and back wall 722 relative to the proximal end portion 708 and the extension member 718 such that the profile of the nasal cleaning cloth 754 may be reduced to be more comfortably inserted into the user's nasal cavity. Optionally, a user may rotate the nasal cleaning cloth 754 and the extension member 718 within the nasal cavity such that movement of the nasal cleaning cloth 754 creates a sweeping-like motion to help remove debris and/or other substances/materials from the nasal cavity. For example, as a user rotates the extension member 718 within the nostril in a first direction, the distal end portion 714 and the unattached portions of the front wall 720 and the back wall 722 may begin to rotate relative to the proximal end portion 708 such that the nasal cleaning cloth 754 wraps or twists around the extension member 718. When the user begins to rotate the extension member 718 within the nostril in a second direction generally opposite the first direction, the distal end portion 714 and the unattached portions of the front wall 720 and the back wall 722 may begin to rotate in the opposite direction relative to the proximal end portion 708 such that the nasal cleaning cloth 754 unwraps or untwists from around the extension member 718. When the extension member 718 is rotated back in the second direction more than it was rotated in the first direction, the distal end portion 714 and the unattached portions of the front wall 720 and the back wall 722 may again begin to wrap or twist the nasal cleaning cloth 754 around the extension member 718. This loosening and tightening of the nasal cleaning cloth 754 about the extension member 718 may help create a sweeping-like motion that pushes and/or pulls debris and/or other materials from the user's nostril. In yet other embodiments, this loosening and tightening of the nasal cleaning cloth 754 about the extension member 718 may help the nasal cleaning cloth 754 moisten the user's nostril by distributing moisturizer or saline solution throughout portions of the nostril.

In yet other embodiments, the distal end portion 714 of the nasal cleaning cloth 754 may be attached to the extension member 718 while the remaining portions of the nasal cleaning cloth 754 are not attached to the extension member 718. In further embodiments, the proximal end portion 708 and the distal end portion 714 of the nasal cleaning cloth 754 may be attached to the extension member 718 while an intermediate portion of the nasal cleaning cloth 754 between the proximal and distal end portions 708, 714 of the nasal cleaning cloth 754 is unattached to the extension member 718. In yet other embodiments, the nasal cleaning cloth 754 may be attached to the extension member 718 along the portion of the length L of the extension member 718 positioned within the internal space 724. In yet other embodiments, the extension member 718 may be attached to the exterior surface of the front wall 720 or the back wall 722.

While the nasal cleaning cloth 754 is described being attached to the extension member 718 via a medical grade adhesive, in other embodiments, the nasal cleaning cloth 754 may be attached to the extension member 718 via buttons, snaps, Velcro® (a hook and loop type closure system), or in any other suitable manner. Moreover, in other embodiments, the nasal cleaning cloth 754 may be removably attached to the extension member 718 such that the nasal cleaning cloth 754 may be removed from the extension member 718 for cleaning or replacement.

The nasal cleaning cloth 754 may include any of the materials described in relation to FIGS. 1 through 6. For example, nasal cleaning cloth 754 may be formed from any suitable material that would allow and/or facilitate removal of debris and/or other materials/substances from inside a nasal cavity and/or moistening of the nasal cavity. In an embodiment, the nasal cleaning cloth 754 may include one or more natural hemp materials. For example, the nasal cleaning cloth 754 comprise a hemp cotton blend fabric. In an embodiment, the hemp cotton blend fabric may exhibit loops and/or tufts on the exterior surface of the front wall 720 and/or the back wall 722. In other embodiments, the hemp cotton blend fabric may exhibit voids, channels, pockets, cross-hatching, and/or other features in and/or on the exterior surface of the front wall 720 and/or the back wall 722. Such a configuration may help dislodge debris from the nasal cavity, and/or provide space to collect and/or hold such debris. In addition, the hemp cotton blend fabric may help provide softness, moisture balancing properties, and/or absorption capabilities to the nasal cleaning fabric 754. Accordingly, the nasal cleaning cloth 754 may be configured to help a user to comfortably moisten the user's nasal cavity. Moreover, the hemp in the nasal cleaning cloth 754 may provide anti-inflammatory, anti-mildew, and/or antimicrobial properties such that the nasal cleaning cloth 754 may help protect a user's nasal cavity from bacteria, fungus, mold, staff infections, and/or the like.

In an embodiment, the hemp cotton blend fabric of the nasal cleaning cloth 754 may include about fifty five percent (55%) hemp and about forty-five percent (45%) cotton. In other embodiments, the hemp cotton blend fabric of the nasal cleaning cloth 754 may include about seventy percent (70%) hemp and about thirty percent cotton (30%); about sixty percent (60%) hemp and about forty percent (40%) cotton; or about fifty percent (50%) hemp and about fifty percent (50%) cotton. In other embodiments, the percentage relationship between the hemp and cotton in the nasal cleaning cloth 754 may be greater or smaller.

In other embodiments, the nasal cleaning cloth 754 may include different materials and/or configurations. For example, the nasal cleaning cloth 754 may comprise a fabric including hemp, cotton, and other natural fibers, recycled PET, silk, cashmere, combinations thereof, or the like. Moreover, in other embodiments, the nasal cleaning cloth 754 may include hemp in combination with other materials such as wool. In yet other embodiments, the distal end portion 714 of the nasal cleaning cloth 754 may include one or more materials such as cotton, fleece, cashmere and the proximal end portion 708 may include a hemp cotton blend fabric. In yet other embodiments, the nasal cleaning cloth 754 may include both plastic and/or polymeric materials and woven fabric materials. For example, the nasal cleaning cloth 754 may include plastic material fibers interwoven with one or more woven or spun hemp and/or cotton materials. Such a configuration may provide both comfort to the user's nostril and effective cleaning due to the rigidity of the plastic material fibers to both dislodge and to cause materials to adhere to the cleaning portion 750.

It will be appreciated that the nasal cleaning cloth 754 may be manufactured or produced in a variety of different ways. For example, in one embodiment, two pieces of fabric may be placed one on top of the other. A selected portion of the fabric pieces may then be attached together in the general shape of the nasal cleaning cloth 754 (e.g., the generally balloon-like shape). For example, a selected portion of the fabric pieces may be stitched or sewn together with thread (i.e., polyester thread) following a path or pattern that generally defines the front wall 720, the back wall 722, the proximal end portion 708, and the distal end portion 714 of the nasal cleaning cloth 754 on the fabric pieces. The stitched path or pattern may also generally define the internal space 724 and the opening 726 of the nasal cleaning cloth 754 between the fabric pieces. With the proximal end portion 708, the distal end portion 714, the front wall 720, the back wall 722, the internal space 724, and the opening 726 generally defined, the nasal cleaning cloth 754 may then be cut away or cut out from the fabric pieces using a laser. In other embodiments, the nasal cleaning cloth 754 may be cut away or cut out from the selected portion of the two pieces of fabric with a mechanical cutting device, a heating device, shearing, or any other means suitable to cut away or cut out the nasal cleaning cloth 754. Moreover, while the fabric pieces are described being attached to together using thread, in other embodiments, the fabric pieces may be attached together using adhesives, mechanical fasteners, heat, or any other suitable attachment means. In other embodiments, the front wall 720 and the back wall 722 of the nasal cleaning cloth 754 may be cut from one, three, four, or any other suitable number of fabric pieces and then attached together to form the nasal cleaning cloth 754. In yet other embodiments, the fabric may be woven, spun, and/or folded to form of the nasal cleaning cloth 754.

Optionally, the nasal cleaning cloth 754 may include a depth indicator element configured to help indicate to a user the depth that the extension member 718 is inserted into a user's nostril. For example, the nasal cleaning cloth 754 may include a visual indicator line (shown in FIG. 9) positioned a selected distance proximal of the distal end of the extension member 718. Accordingly, when the cleaning cloth 754 is positioned or being used within the user's nostril and the visual indicator line on the nasal cleaning cloth 754 approaches or passes into the nostril, the visual presence or absence of the indicator line may indicate to the user that the depth of the extension member 718 within the nostril is at or near a selected depth. For instance, the indicator line may be positioned at about one-half inch below the distal end of the extension member 718 such that a user knows not to insert the extension member 718 more than one-half inch into the user's nostril. Such a configuration may encourage safe usage of the apparatus 700 by helping to limit over insertion of the apparatus 700 within a user's nostril. In other embodiments, the indicator element may be configured and positioned to indicate a depth of other portions of the apparatus 700 within the nostril such as the nasal cleaning cloth 754 or a head portion. In other embodiments, the indicator element may comprise an acoustic indicator element or light indicator element associated with a sensor configured to detect the depth of the extension member 718 within the nostril.

Moreover, while cleaning portion 750 is illustrated without a separate head portion, in other embodiments, cleaning portion 750 may include a head portion similar to any of the head portions described in relation to FIGS. 1 through 6. For example, extension member 718 may be attached to a head portion positioned within the internal space 724 of the nasal cleaning cloth 754.

FIG. 9 illustrates a perspective view of the nasal cleaning apparatus 700 being inserted in a nostril. In an embodiment, when a user desires to use the apparatus 700 to clean a nostril and/or nasal cavity, a user can hold grip portion 710 with the user's hand, fingers, and/or finger tips. The user may then twist or gently wrap at least a portion of the nasal cleaning cloth 754 around the extension member 718 such that the cleaning portion 750 of the apparatus 700 exhibits a reduced or lower profile and/or size. With the nasal cleaning cloth 754 twisted around the extension member 718, the user may then insert at least a portion of the nasal cleaning cloth 754 and the extension member 718 into the nostril or nasal cavity. The nostril or nasal cavity may be of the user or another person such as a medical patient. Optionally, before twisting and/or inserting the nasal cleaning cloth 754 into the nostril or nasal cavity, the user may moisten the nasal cleaning cloth 754 with warm water, saline solution, ointment, or other suitable substance. Accordingly, the nasal cleaning apparatus 700 may be used to moisten the nostril or nasal cavity in addition to or as an alternative to cleaning the nostril or nasal cavity. For example, a user who is an oxygen recipient or one that lives in a dry climate may use the nasal cleaning apparatus 700 to moisten the user's nostril in order to help prevent infection, inflammation, disease, nose bleeds, dry nostrils, or the like.

With at least a portion of the nasal cleaning cloth 754 positioned within the nostril and/or nasal cavity, the user may rotate the apparatus 700 in a circular motion in a first and/or second direction. As shown, rotation of the apparatus 700 may cause at least a portion of the extension member 718 and/or nasal cleaning cloth 754 to rotate within the nostril and/or nasal cavity. Motion of the nasal cleaning cloth 754 within the nostril and/or nasal cavity may help moisten or loosen, clean and/or remove debris and other materials from the nostril and/or nasal cavity. For example, the twisting and/or untwisting of the nasal cleaning cloth 754 within the nostril and/or nasal cavity, may create a sweeping-type motion with the nasal cleaning cloth 754 to help clean and/or dislodge debris from the nostril and/or nasal cavity. Optionally, in addition to rotating the nasal cleaning cloth 754 in a circular motion, the user may also gently move the nasal cleaning cloth 754 up and down using the handle 712 to further moisten, clean, and/or remove debris and/or other materials from the nostril and/or nasal cavity. When a user is finished moistening and/or cleaning, the user may remove the apparatus 700 from the nostril and/or nasal cavity. In some embodiments, removing the apparatus from the nostril and/or nasal cavity may help pull debris and/or other materials from the nostril and/or nasal cavity with the nasal cleaning cloth 754. In an embodiment, the user may then wash the nasal cleaning cloth 754 such that the apparatus 700 may be used at another time. In other embodiments, the user may safely discard the apparatus 700. While the above exemplary method is described with reference to nasal cleaning apparatus 700, it will be appreciated that any or all of the foregoing steps may be performed using any of the nasal cleaning apparatuses and/or kits disclosed herein. Moreover, it will be appreciated that the nasal cleaning apparatuses described herein may be configured for use with other body parts such as the mouth, ears, or the like.

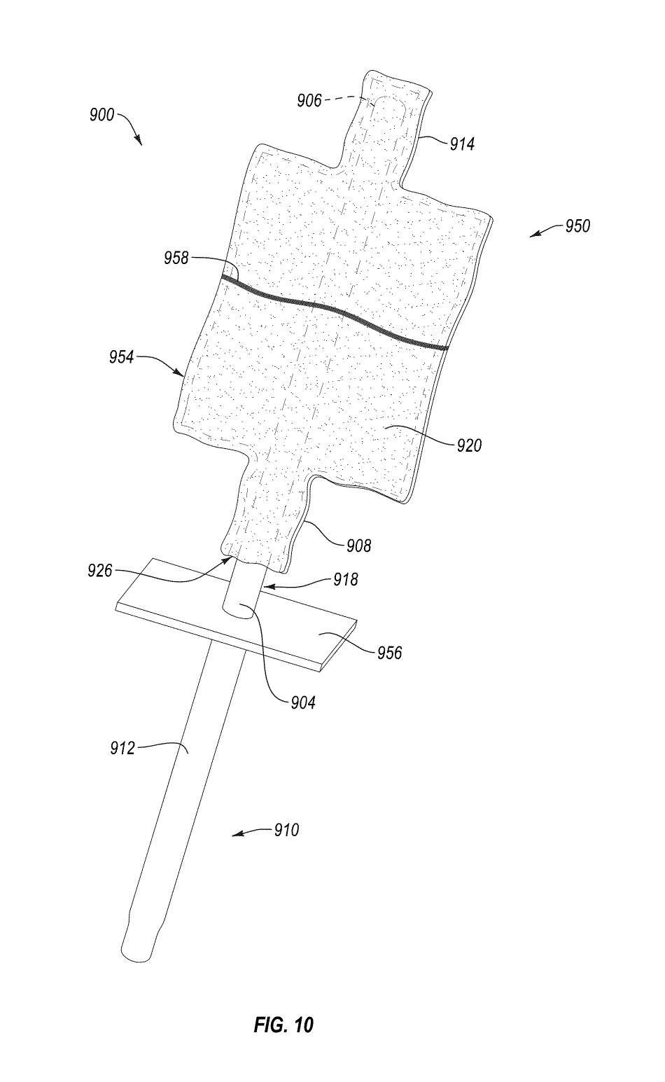

FIG. 10 illustrates another embodiment of another nasal cleaning apparatus 900. The nasal cleaning apparatus 900 may be similar in many respects to the nasal cleaning apparatuses 100, 300, and/or 700 previously described above in relation to FIGS. 1 through 8. Therefore, in the interest of brevity, the components and features of apparatus 900 and apparatuses 100, 300, and 700 that correspond to each other have been provided with the same or similar reference numerals, and an explanation thereof will not be repeated. However, it should be noted that the principles of the apparatus 900 may be employed with any of the embodiments described in relation to FIGS. 1 through 8 and/or vice versa.

Nasal cleaning apparatus 900 is similar to nasal cleaning apparatus 700 in many respects. For example, nasal cleaning apparatus 900 includes a grip portion 910 and a cleaning portion 950. The grip portion 910 may include a handle 912 and an extension member 918 extending from handle 912. A distal end of handle 912 may include a shield member 956 configured to help limit saline solution, body fluids, or other substances and/or materials from running down the extension member 918 onto a user's fingers and/or hand. As shown, the shield member 956 may exhibit a generally rectangular geometric shape having a cross-sectional area greater than a cross-sectional area of at least a portion the handle 912. Accordingly, the shield member 956 may help catch and/or divert substances and/or materials traveling from the user's nostril before such substances and/or materials contact a user's fingers and/or hands on the handle 912.

Similar to the nasal cleaning apparatus 700, the cleaning portion 950 may be directly or indirectly connected to the grip portion 910. As shown, the cleaning portion 950 may comprise a nasal cleaning cloth 954 attached to the extension member 918. The nasal cleaning cloth 954 may include any of the materials described in relation to FIGS. 1 through 8 and may exhibit any suitable shape and/or configuration. For example, the nasal cleaning cloth 954 may exhibit a generally winged body-like shape including a smaller rectangular distal end portion 914, a smaller rectangular proximal end portion 908, and a larger rectangular main body. The larger rectangular main body may include wing-like side portions extending laterally beyond the proximal and distal end portions 908, 914. Such a configuration may help the nasal cleaning cloth 954 to sweep and/or clean debris and/or other materials from the user's nostrils. Of course, it will be appreciated that in other embodiments, the nasal cleaning cloth 954 may exhibit other suitable shapes.

In the illustrated embodiment, the nasal cleaning cloth 954 may also comprise a sock-like body similar to nasal cleaning cloth 754. For example, the nasal cleaning cloth 954 may include a front wall 920 extending between the distal end portion 914 and the proximal end portion 908, and a back wall (not shown). Both the front wall 920 and the back wall may include an interior surface and an exterior surface. In an embodiment, the front wall 920 and the back wall may be attached along at least a portion of the periphery of the walls such that the interior surfaces of the front wall 920 and the back wall form an internal space (not shown). Another portion of the periphery of the walls may form an opening 926 at the proximal end portion 908 of the nasal cleaning cloth 954 in communication with the internal space. The internal space may be accessible through the opening 926. The front wall 920 and the back wall may be connected together in any suitable manner. For example, the front wall 920 may be connected to the back wall via adhesives, stitching, mechanical fasteners, combinations thereof, or by any other suitable means.

The opening 926 may be in communication with the internal space such that the extension member 918 of grip portion 910 may at least partially be extended through opening 926 into the internal space. In the illustrated embodiment, the extension member 918 may extend substantially between the proximal end portion 908 and the distal end portion 914 within the internal space. In other embodiments, the extension member 918 may extend more or less within the internal space.

The nasal cleaning cloth 954 may be connected to the extension member 918 in any suitable manner. For example, in the illustrated embodiment, the distal end portion 914 of the nasal cleaning cloth 954 may be attached to a distal end portion 914 of the extension member 918 and the proximal end portion 908 of the nasal cleaning cloth 954 may be attached to the extension member 918. Such a configuration may allow the unattached rectangular body portion of the nasal cleaning cloth 954 to be rotatable and/or movable relative to the extension member 918 and/or the proximal and distal end portions 908, 914 of the nasal cleaning cloth 954. Accordingly, a user may twist and/or untwist the nasal cleaning cloth 954 about the extension member 918. Moreover, debris removal and/or cleaning of the user's nostril may be enhanced due to the sweeping of the wing-like side portions of the main body portion on the nasal cleaning cloth 954. In other embodiments, the nasal cleaning cloth 954 may be connected to the extension member 918 in other suitable configurations.

Optionally, the nasal cleaning cloth 954 may include a depth indicator 958 configured to help indicate to a user the depth the extension member 918 is inserted in a user's nostril. The depth indicator 958 may comprise a visual indicator line positioned at a selected distance proximal of the distal end 906 of the extension member 918. Accordingly, when the cleaning cloth 954 is positioned or being used within the user's nostril and the depth indicator 958 on the nasal cleaning cloth 954 approaches or passes into the nostril, the visual presence or absence of the depth indicator 958 may indicate to the user that the depth of the extension member 918 within the nostril is at or near a selected depth.

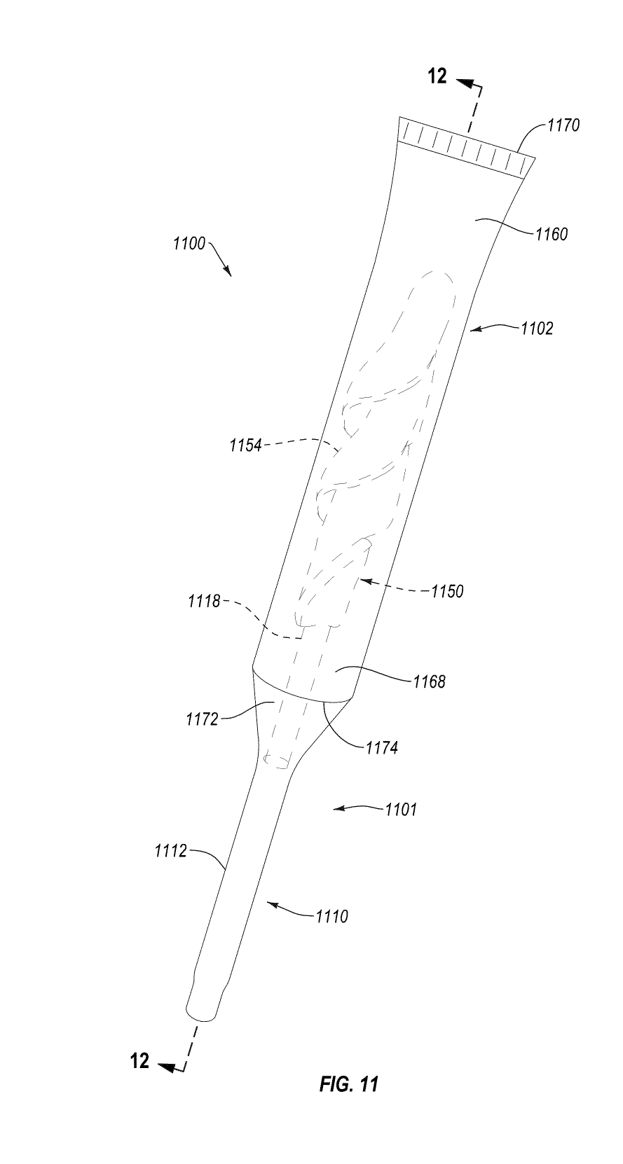

FIG. 11 illustrates a nasal cleaning kit 1100 according to an embodiment. Nasal cleaning kit 1100 may include a nasal cleaning apparatus 1101 and a storage tube 1102. The nasal cleaning apparatus 1101 may be configured as any of the nasal cleaning apparatuses disclosed herein. For example, nasal cleaning apparatus 1101 may include a grip portion 1110 and a cleaning portion 1150. The grip portion 1110 may include a handle 1112 and an extension member 1118 extending from handle 1112. The cleaning portion 1150 may comprise a nasal cleaning cloth 1154 attached to the extension member 1118.

The storage tube 1102 may be configured to selectively store the cleaning portion 1150 of the nasal cleaning apparatus 1101. For example, in an embodiment, the storage tube 1102 may exhibit a generally elongated cylindrical shape including an exterior surface 1160 and an inner surface 1162 defining a lumen 1164 within the storage tube 1102. The lumen 1164 may be configured to selectively receive and store at least the cleaning portion 1150 of the nasal cleaning apparatus 1101. As illustrated, the nasal cleaning cloth 1154 may be compressed within the lumen 1164 in a storage configuration. Upon removal of the nasal cleaning cloth 1154 from the lumen 1164 of the storage tube 1102, the nasal cleaning cloth 1154 may move toward a released configuration, wherein the nasal cleaning cloth 1154 expands to a resting position.

In other embodiments, the storage tube 1102 may exhibit a generally tubular rectangular shape, a generally tubular elliptical shape, a generally tubular conical shape, or any other suitable shape. In an embodiment, the storage tube 1102 may exhibit an exterior shape that is different than an interior shape of the lumen 1164 of the storage tube 1102. For example, the storage tube 1102 may include a generally cylindrical lumen 1164 and a generally rectangular exterior surface 1160. The storage tube 1102 may include one or more plastic materials, polymeric materials, organic materials, rubber materials, polyethylene materials, metals, metal alloys, combinations thereof, or the like.

In the illustrated embodiment, the storage tube 1102 may include an opening 1166 at a proximal end portion 1168 of the storage tube 1102 that is in communication with the lumen 1164. The opening 1166 may be sized and/or configured such that the nasal cleaning cloth 1154 and at least a portion of the extension member 1118 of the nasal cleaning apparatus 1101 may pass through the opening 1166 into the lumen 1164 of the storage tube 1102.

In the illustrated embodiment, the storage tube 1102 may further include a sealed distal end portion 1170 opposite the opening 1166. The sealed distal end portion 1170 may be sealed by any suitable means such as, for example, heat sealing, plastic welding, adhesives, or the like. In other embodiments, the sealed distal end portion 1170 may be omitted. For example, the storage tube 1102 may be integrally formed into a cylindrical shape including a planar distal end portion 1170. In an embodiment, the storage tube 1102 may exhibit an outer cross-sectional area that is greater than an outer cross-sectional area of at least a portion of the handle 1112. Moreover, in other embodiments, the cross-sectional area of the conical distal portion of the handle 1112 may increase to join the storage tube 1102.

The storage tube 1102 may be removably attached to and/or selectively detachable from the nasal cleaning apparatus 1101 in a variety of different ways. For example, in the illustrated embodiment, the conical distal portion of the handle 1112 may function as a connection portion 1172 between the nasal cleaning apparatus 1101 and the proximal end portion 1168 of the storage tube 1102. In an embodiment, the storage tube 1102 and the nasal cleaning apparatus 1101 may be connected together via a sealed connection 1174 such that the nasal cleaning portion 1150 of the nasal cleaning apparatus 1101 is substantially sealed and/or positioned within the lumen 1164 of the storage tube 1102. A breakaway recess 1176 may be formed in the sealed connection 1174 that extends from an exterior surface of the sealed connection 1174 toward the interior surface 1162 of the storage tube 1102. In other embodiments, the storage tube 1102 and the nasal cleaning apparatus 1101 may comprise a single member and the breakaway recess 1176 may be formed in the exterior surface 1160 of the storage tube 1102 or the connection portion 1172. In yet other embodiments, the breakaway recess 1176 may be formed in any suitable portion of the storage tube 1102 and/or nasal cleaning apparatus 1101. In an embodiment, the breakaway recess 1176 may circumscribe the sealed connection 1174, the storage tube 1102, and/or the connection portion 1172. In other embodiments, the breakaway recess 1176 may circumscribe only a portion of the sealed connection 1174, the storage tube 1102, and/or the connection portion 1172. In other embodiments, the storage tube 1102 and the nasal cleaning apparatus 1101 may be selectively detachable and attached to one another via magnets, an interference fit connection, adhesives, mechanical fasteners, combinations thereof, or the like.

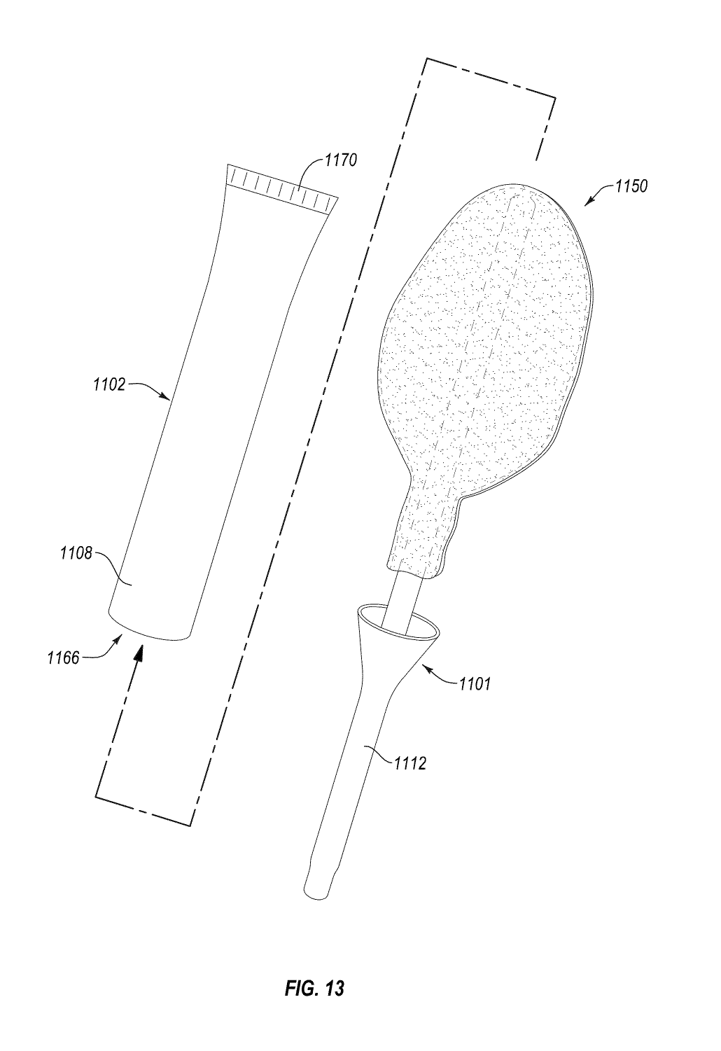

To separate the storage tube 1102 from the nasal cleaning apparatus 1101, a user may grasp the storage tube 1102 in one hand and the handle 1112 of the nasal cleaning apparatus 1101 in the other hand. The user may then bend or squeeze the nasal cleaning kit 1100 at the breakaway recess 1176 which creates one or more stresses at the breakaway recess 1176. At least a portion of the nasal cleaning kit 1100 may break or snap open at the breakaway recess 1176. Referring to FIG. 13, at least a portion of the storage tube 1102 may be severed from the handle 1112 of the nasal cleaning apparatus 1101. The nasal cleaning apparatus 1101 may then be removed to expose the cleaning portion 1150 or nasal cleaning cloth 1154.

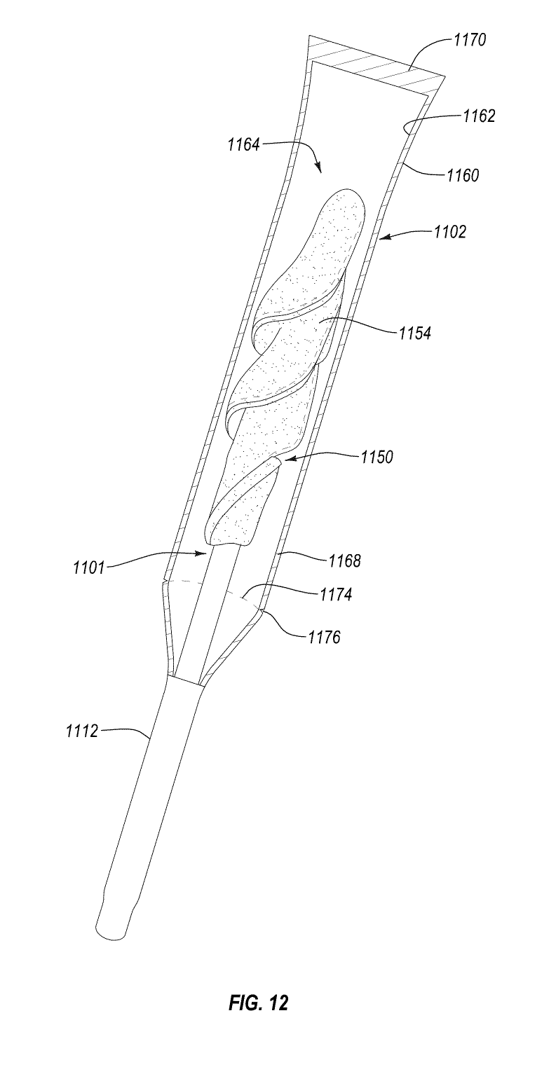

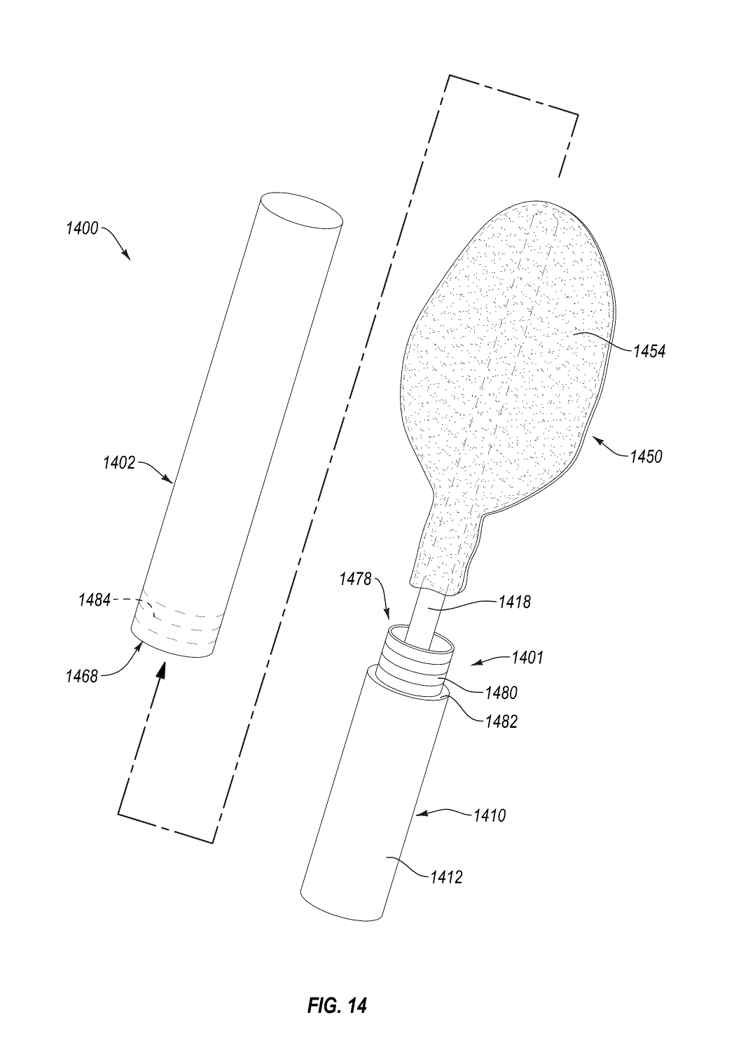

Referring now to FIG. 14, a nasal cleaning kit 1400 will be described according to another embodiment. The nasal cleaning kit 1400 may be similar in many respects to the nasal cleaning kit 1100 previously described in relation to FIGS. 11 through 13. Therefore, in the interest of brevity, the components and features of nasal cleaning kit 1400 and nasal cleaning kit 1100 that correspond to each other have been provided with the same or similar reference numbers, and an explanation thereof will not be repeated. However, it should be noted that the principles of the nasal cleaning kit 1400 may be employed with any of the embodiments described in relation to FIGS. 1 through 13 and/or vice versa.

Nasal cleaning kit 1400 may include a nasal cleaning apparatus 1401 and a storage tube 1402. The nasal cleaning apparatus 1401 may be configured as any of the nasal cleaning apparatuses disclosed herein. For example nasal cleaning apparatus 1401 may include a grip portion 1410 and a cleaning portion 1450. The grip portion 1410 may include a handle 1412 and an extension member 1418 extending from the handle 1412. The cleaning portion 1450 may comprise a nasal cleaning cloth 1454 attached to the extension member 1418. As illustrated, the storage tube 1402 and the handle 1412 may have generally similar outer cross-sectional areas. In other embodiments, the storage tube 1402 and/or the handle 1412 may have different cross-sectional areas. For example, the storage tube 1402 may have an outer and/or inner cross-sectional area that is greater than an outer and/or inner cross-sectional area of the handle 1412 or vice versa. In other embodiments, the storage tube 1402 and/or the handle 1412 may exhibit a cross-sectional area that varies along a respective length thereof.

In the illustrated embodiment, a distal end portion 1478 of the handle 1412 may include a male threaded portion 1480 and a shoulder 1482 proximate the male threaded portion 1480. A proximal end portion 1468 of the storage tube 1402 may include a female threaded portion 1484 on the interior surface of the storage tube 1402 configured to correspond to the threaded portion 1480 of the handle 1412. Accordingly, the storage tube 1402 may be selectively attached and detached from the nasal cleaning apparatus 1401 by rotating the storage tube 1402 and/or the nasal cleaning apparatus 1401 relative to the other. For example, the cleaning portion 1450 of the nasal cleaning apparatus 1401 may be positioned within a lumen (not shown) of the storage tube 1402 and the storage tube 1402 may be threaded onto the handle 1412 of the nasal cleaning apparatus 1401 until the proximal end portion 1468 of the storage tube 1412 engages the shoulder 1482 of the handle 1412. In other embodiments, the threaded portions may be omitted and the storage tube 1402 and the nasal cleaning apparatus may be attached to one another and selectively detachable via an interference fit type connection. In yet other embodiments, the storage tube 1402 and the nasal cleaning apparatus 1401 may be selectively attached to one another via magnets, a press-fit connection, adhesives, mechanical fasteners, combinations thereof, and/or by any other suitable means.

The various embodiments disclosed and contemplated herein thus allow users to prevent the onset of illness. In other cases, the user can remove materials to alleviate suffering from allergies, illness and/or asthma. The removal of these materials facilitates the breathing of cleaner, fresher air. In other cases, the user can use the nasal cleaning apparatus to moisten the nasal cavity to help prevent the onset of disease, allergy symptoms, and/or germs. The ease of use allows a user to use the nasal cleaning apparatus daily or as needed to obtain the proper care and cleaning of the nasal cavity through the nostrils. Accordingly, according to aspects of the present invention, a user of the nasal cleaning apparatus can gently and effectively moisten and/or clean his or her nostrils to aid in the prevention or remediation of breathing problems and/or of the flu and chronic sinus problems. The product is further safe and easy to use for people of all ages.

The present invention may be embodied in other specific forms without departing from its spirit or essential characteristics. The described embodiments are to be considered in all respects only as illustrative and not restrictive.

What is claimed is:

1. A nasal cleaning apparatus comprising:
a nasal cleaning portion configured to be inserted into a nostril to help moisten and/or clean the nostril, said nasal cleaning portion comprising:
a proximal end portion and a distal end portion;
a front wall extending between said proximal end portion and said distal end portion,
a back wall, said back wall being attached to said front wall along at least a portion of the periphery of said front wall and said back wall;
an internal space at least partially defined between said front wall and said back wall;
an opening in said proximal end portion, said opening being in communication with said internal space;
a grip portion comprising:
a handle including a distal end portion and a proximal end portion;
an extension member extending from said distal end portion of said handle, through said opening of said nasal cleaning portion, and into said internal space of said nasal cleaning portion.

2. The nasal cleaning apparatus of claim 1, wherein said proximal end portion of said nasal cleaning portion is attached to said extension member and said distal end portion of said nasal cleaning portion is not attached to said extension member.

3. The nasal cleaning apparatus of claim 2, wherein at least said distal end portion of said nasal cleaning portion is moveable and/or rotatable relative to said extension member and said proximal end portion of said nasal cleaning portion.

4. The nasal cleaning apparatus of claim 2, wherein at least a portion of said nasal cleaning portion is configured to selectively twist or wrap around at least a portion of said extension member.

5. The nasal cleaning apparatus of claim 1, wherein said nasal cleaning portion further comprises an intermediate portion extending between said distal end portion and said proximal end portion, and wherein said proximal end portion of said nasal cleaning portion is attached to said extension member and said distal end portion of said nasal cleaning portion is attached to said extension member, and wherein said intermediate portion of said nasal cleaning portion is moveable and/or rotatable relative to said proximal end portion and said distal end portion of said nasal cleaning portion.

6. The nasal cleaning apparatus of claim 1, wherein said nasal cleaning portion includes an indicator element configured to indicate a depth of said extension member or said nasal cleaning portion inserted into a nostril of a user.

7. The nasal cleaning apparatus of claim 1, wherein said nasal cleaning portion comprises a hemp cotton blend fabric.

8. The nasal cleaning apparatus of claim 7, wherein said hemp cotton blend fabric includes one or more loops on at least one surface thereof.

9. The nasal cleaning apparatus of claim 1, wherein said extension member comprises an elongate tubular member.

10. The nasal cleaning apparatus of claim 1, wherein said extension member includes a length defined between a distal end portion of said handle and a distal end portion of said extension member, and wherein at least about twenty percent, at least about forty percent, at least about sixty percent, or at least about eighty percent of said length of said extension member is positioned within said internal space of said nasal cleaning portion.

11. The nasal cleaning apparatus of claim 1, wherein said extension member includes a length defined between a distal end portion of said handle and a distal end portion of said extension member, and wherein between about forty percent and about eighty percent of said length of said extension member is positioned within said internal space of said nasal cleaning portion.

12. The nasal cleaning apparatus of claim 10, wherein said length of said extension member is between about two inches and five inches.

13. The nasal cleaning apparatus of claim 7, wherein at least a portion of said extension member positioned within said internal space of said nasal cleaning portion includes one or perforations in communication with a lumen extending through said extension member.

14. The nasal cleaning apparatus of claim 1, wherein said internal space of said nasal cleaning portion is configured to house one or more substances to be provided to a nostril of a user through one or more pores or voids formed in said nasal cleaning portion in communication with said internal space.

15. The nasal cleaning apparatus of claim 1, further comprising a storage tube attached to said handle, said storage tube including a lumen extending at least partially along a longitudinal axis of said storage tube, said lumen being configured to selectively receive and store at least said nasal cleaning portion, wherein said storage tube is selectively detachable from said handle.

16. A nasal cleaning kit comprising:
a nasal cleaning apparatus including:
a nasal cleaning portion comprising a sock-like body including a proximal end portion and a distal end portion, an internal space within said sock-like body, and an opening formed in said proximal end portion, said opening being in communication with said internal space; and
a grip portion including a handle and an extension member connected to said handle, wherein at least a portion of said extension member extends through said opening of said nasal cleaning portion into said internal space of said nasal cleaning portion;
a storage tube removably attached to said handle of said grip portion, said storage tube including a lumen extending at least partially through said storage tube, wherein said lumen is configured to selectively receive and store at least said nasal cleaning portion of said nasal cleaning apparatus.

16. The nasal cleaning kit of claim 16, wherein said storage tube and said handle of said grip portion are connected via a sealed connection.

17. The nasal cleaning kit of claim 16, wherein said storage tube and said handle are threadedly connected to one another.

18. The nasal cleaning kit of claim 16, wherein said storage tube and said nasal cleaning apparatus are attached to each other via a connection including a breakaway recess extending from an exterior surface of said connection toward an interior surface of said connection.

19. A method for moistening and/or cleaning a nasal cavity, the method comprising:
providing a nasal cleaning apparatus comprising:
a nasal cleaning portion configured to be inserted into a nasal cavity to moisten and/or clean the nasal cavity, said nasal cleaning portion comprising:
a proximal end portion and a distal end portion;
a front wall extending between said proximal end portion and said distal end portion,
a back wall, said back wall being attached to said front wall along a portion of the periphery of said front wall and/or said back wall;
an internal space at least partially defined between said front wall and said back wall;
an opening in said proximal end portion, said opening being in communication with said internal space;
a grip portion comprising:
a handle including a distal end portion and a proximal end portion;
an extension member extending from said distal end portion of said handle, through said opening of said nasal cleaning portion, and into said internal space of said nasal cleaning portion.
grasping said grip portion with a hand and/or fingers of the user;
twisting said nasal cleaning portion about said extension member;
inserting at least a portion of said nasal cleaning portion into the nasal cavity;
rotating said nasal cleaning portion with said extension member to at least partially moisten and/or clean the nasal cavity; and
removing said nasal cleaning portion from the nasal cavity.

20. The method of claim 19, further comprising:
moistening said nasal cleaning portion with at least one of water, moisturizer or saline solution before inserting said at least a portion of said nasal cleaning portion into the nasal cavity.
